(19) United States
(12) Patent Application Publication
(10) Pub. No.: US 2003/0167033 A1
(43) Pub. Date: Sep. 4, 2003

(54) Systems and methods for photodynamic therapy

(76) Inventors: James Chen, Clyde Hill, WA (US); Julene Christophersen, Sammamish, WA (US); Nick Yeo, Surrey (GB); Greg Heacock, Cammas, WA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(21) Appl. No.: 10/351,730

(22) Filed: Jan. 23, 2003

Related U.S. Application Data
(60) Provisional application No. 60/351,460, filed on Jan. 23, 2002.

(51) Int. Cl. A61N 5/06

(52) U.S. Cl. 604/20; 607/88

(57) ABSTRACT

Systems and methods for performing photodynamic therapy wherein a photoreactive agent is delivered locally and activated with an external non-invasive energy source are provided. In one embodiment, a low energy light source is used to initiate fluorescence in target tissue containing photoreactive agent. The characteristic fluorescence of the abnormal target tissue is used to generate a map that is then used to direct targeted activation energy to the target tissue without collateral damage to healthy tissue.

### REFERENCE TO PRIORITY DOCUMENTS

This application claims priority of co-pending U.S. Provisional Patent Application Serial No. 60/351,460, entitled “Systems And Methods For Photodynamic Therapy”, filed Jan. 23, 2002. Priority of the aforementioned filing date is hereby claimed, and the disclosure of the aforementioned U.S. Provisional Patent Application is hereby incorporated by reference in its entirety. Also incorporated by reference in its entirety is co-pending Internation Patent Cooperation Treaty (PCT) Patent Application No. ______ (Attorney Docket No. 25886-0052PC), entitled “Systems And Methods For Photodynamic Therapy”, which is filed on the same date as the instant application.

### FIELD OF THE INVENTION

Provided herein are methods of photodynamic therapy and diagnosis. In particular, methods of photodynamic therapy using non-invasive transcutaneous or transocular light delivery are provided.

### BACKGROUND OF THE INVENTION

Photodynamic therapy is a process whereby light of a specific wavelength or waveband is directed to tissues undergoing treatment or investigation that have been rendered photosensitive through the administration of a photoreactive or photosensitizing agent. The objective of the intervention may be either diagnostic, where the wavelength or waveband of light is selected to cause the photoreactive agent to fluoresce, thus yielding information about the tissue without damaging the tissue, or therapeutic, where the wavelength of light delivered to the photosensitive tissue under treatment causes the photoreactive agent to undergo a photochemical interaction with oxygen in the tissue under treatment that yields free radical species, such as singlet oxygen, causing local tissue lysing or destruction.

Photodynamic therapy (PDT) has proven to be very effective in destroying abnormal tissue such as cancer cells. In this therapy, a photoreactive agent having a characteristic light absorption waveband is first administered to the patient, typically either orally or by injection. Abnormal tissue in the body is known to selectively absorb certain photoreactive agents to a much greater extent than normal tissue, e.g., tumors of the pancreas and colon may absorb two to three times the volume of these agents, compared to normal tissue. Even more effective selectivity is achieved using a photoreactive agent that is bound to an antibody, which links with antigens on targeted cells. However, some of the undesirable side effects of systemic delivery of photoreactive agents to a patient can include skin photosensitivity, which can result in serious burns resulting from exposure to sunlight, back pain, headache, injection site complications such as extravasation and rash, and allergic reactions to the photoreactive agent.

Once the cancerous or abnormal tissue has absorbed or linked with the photoreactive agent as discussed above, the abnormal or cancerous tissue can then be destroyed by administering light of an appropriate wavelength or waveband corresponding to the absorption wavelength or waveband of the photoreactive agent. To administer PDT to internal cancerous lesions that are not accessible through a natural body orifice, a fiber optic probe is typically inserted either through a needle or through a surgically created opening. When the internal treatment site is accessible through natural body orifices, an endoscope is used to visualize the lesion and accurately direct the light therapy administered to the treatment site. The invasive placement of an optical fiber probe or endoscope at an internal treatment site exposes a patient to potential risks associated with bleeding, infection, and the use of anesthesia and sedation. In addition, these potential limitations can limit the amount of light exposure time for the tissue which has absorbed the photoreactive agent. What has been needed is a system and method of performing PDT that allows for the use of non-systemic delivery of a photoreactive agent to a patient and non-invasive photoactivation of the target tissue.

In addition, one of the problems with administering light therapy to an internal treatment site with an externally applied light source can relate to the difficulty in accurately directing the light through the overlying tissue, since the disposition of the internal treatment site is normally not visually apparent to the medical practitioner. However, it is possible to employ various imaging systems to identify the location of abnormal tissue within a patient's body, including its depth below the dermal layer. Suitable imaging systems capable of imaging soft tissue structures to locate internal diseased sites include ultrasound probes and angiography. By viewing the images of the patient's internal body structure it is possible to determine an appropriate position, direction, and depth at which to focus light of an appropriate waveband at a position on the patient's skin. If the light is not accurately directed, damage may occur to healthy tissue collateral to the lesion site, such as in retinal therapy commensurate with treatment of age-related macular degeneration (AMD).

Therefore, what has also been needed is a system and method to target non-invasive externally delivered photoactivation energy or light specifically to the target lesion so as to minimize collateral damage to healthy tissue.

### SUMMARY

Systems and methods for treating neoplastic, neovascular and hypertrophic diseases are provided. In one embodiment, systems and methods for performing photodynamic therapy using localized delivery of a photoreactive agent to target tissue are provided. The photoreactive agent is photoactivated by a non-invasive light source located external to the patient's body. In this way, the need for an infusionist to systemically infuse the photoreactive agent, resulting photosensitivity of the patient, and the need for a large amount of photoreactive agent is avoided. In addition, the potential trauma, infection and limited activation time caused by an invasive light delivery system are avoided.

In certain embodiments, the methods provided herein include performing photodynamic therapy on a patient which includes locally delivering a photoreactive agent having an activation wavelength range to target tissue of a patient. The photoreactive agent is then photoactivated with electromagnetic radiation having a wavelength within the activation wavelength range. The electromagnetic radiation travels from outside the patient's body to the target tissue within the patient's body. In certain embodiments, the photoreactive agent is locally delivered to the target tissue by injection through a hypodermic needle, the disposition of a photoreactive agent depot within or adjacent the target tissue, injection through a coronary delivery catheter for coronary indications or injection through a urinary delivery catheter for prostate or urinary indications. Optionally, the target tissue is allowed to absorb a clinically beneficial amount of the photoreactive agent prior to exposure to the electromagnetic radiation.

Another embodiment includes a method of performing photodynamic therapy on an eye of a patient including administering a photoreactive agent to the patient's body and optionally allowing the photoreactive agent to absorb into at least a portion of the patient's retina. The patient's retina is then illuminated with a fluorescence generating light so that the photoreactive agent in the patient's retina fluoresces and emits fluorescent light. The fluorescent light emitted from the patient's retina is then detected with a fluorescence detector capable of spatially segregating the location of a point source of fluorescent light from different points in the patient's retina and storage of fluorescent response data from various points of the patient's retina. A processor then processes the fluorescence response date and generates a map of at least a portion of the patient's retina so as to-create a map of the fluorescence response of the patient's retina indicating at least one location of abnormality on the patient's retina. Thereafter, photoreactive light is delivered to the patient's retina and is targeted to the at least one location of abnormality on the patient's retina. In some embodiments, the photoreactive agent is delivered to the patient's retina locally by placing a contact disk on the cornea of the patient's eye, application of the photoreactive agent to the patient's eye in conjunction with ultrasonic energy which facilitates permeation of the photoreactive agent into the eye and gas jet injection of the photoreactive agent adjacent the sclera of the patient's eye.

Another embodiment includes a system for performing photodynamic therapy on a patient's retina including a source of fluorescence generating light configured to illuminate the retina of the patient, a fluorescence detector configured to detect fluorescent light emanating from the retina of the patient and a source of photoactivating light configured to deliver photoactivating light to the patient's retina. A processor is programmed to accumulate, store and analyze fluorescence response data from the fluorescence detector in response to fluorescent light from the patient's retina. The processor can then generate a map of the patient's retina based on the fluorescence data indicating locations of tissue abnormality and thereafter direct light from the source of photoactivating light so as to be specifically targeted to the locations of tissue abnormality in the patient's retina. By specifically targeting the photoactivating light to the locations of tissue abnormality, collateral damage to surrounding tissue is minimized or avoided completely.

Another embodiment includes a device for performing photodynamic therapy on the eye of a patient, the device including an elongate arm and a photoactivating light source. At least a portion of the arm follows a curvature that substantially conforms to the curvature of the eye. The photoactivating light source emits light along a light path and the light source is positioned at a distal end of the elongate arm. The elongate arm is sized to be positioned adjacent an outer surface of the eye such that a target portion of the eye is positioned in the light path.

Another embodiment includes a device for delivering a photoreactive agent to the eye of a patient. The device includes a hypodermic needle, wherein at least a portion of the needle follows a curvature that substantially conforms to the curvature of the eye, and wherein the photoreactive agent can be dispensed from a distal end of the needle. The device also includes a sheath that at least partially surrounds the needle, wherein the sheath follows a curvature that substantially conforms to the curvature of the eye.

### BRIEF DESCRIPTION OF THE DRAWINGS

FIG. 1 shows a diagrammatic view of a patient with a hypodermic needle disposed within target tissue and a photoactivating LED array disposed externally to the patient's chest adjacent the target tissue.

FIG. 2 is a cross sectional view of patient tissue showing target tissue with the tip of a hypodermic needle and a photoreactive agent depot disposed therein.

FIG. 3 is an enlarged diagrammatic view of the LED array of FIG. 1 disposed outside the dermal layer adjacent target tissue with light from the LED array penetrating the dermal layer and impinging on the target tissue.

FIG. 4 shows a patient with a coronary delivery catheter disposed within a coronary artery and LED array outside the patient's chest adjacent the target tissue within the coronary artery.

FIG. 5 is an enlarged view of FIG. 4 showing the patient's heart and coronary artery with the coronary delivery catheter disposed within the coronary artery adjacent target tissue.

FIG. 6 shows the balloon portion of the coronary delivery catheter of FIGS. 4 and 5.

FIG. 7 is a sectional view of the urinary anatomy of a patient having a urinary delivery catheter disposed within the patient's urethra and an LED array configured to activate a photoreactive agent disposed external to the patient's body adjacent the target tissue.

FIG. 8 is an elevational view in longitudinal section of the urinary delivery catheter of FIG. 7.

FIG. 9 is a sectional view of a patient's eye with a thin hypodermic needle disposed within the vitreous humor of the patient's eye adjacent the retina for delivery of a photoreactive agent. Also shown are two photoreactive drug depots disposed behind the patient's eye.

FIG. 10 is a sectional view of a patient's eye showing a contact disk disposed on the cornea of the eye.

FIG. 11 is a sectional view of a patient's eye with a distal end of a gas jet injector disposed between the eye and eye socket of the patient for gas jet delivery of a photoreactive agent to the tissue behind the eye adjacent the target tissue of the patient's retina.

FIG. 12 is a sectional view of a patient's eye with a distal end of an ultrasonic probe for delivery of a photoreactive compound disposed on the sclera of the patient's eye.

FIG. 13 is a sectional view of a patient's eye that has been dosed with a photoreactive agent.

FIG. 14 shows the retina of the patient's eye shown in a cross sectional view of the eye of FIG. 13 taken along lines 14-14 of FIG. 13, and indicating the affected area of the retina due to age-related macular degeneration.

FIG. 15 shows the retina of the patient's eye shown in a cross sectional view of the eye of FIG. 13 taken along lines 14-14 of FIG. 13, and indicating the affected area of the retina due to diabetic retinopathy.

FIG. 16 is a diagrammatic view of a system for performing photodynamic therapy on a patient's retina having features indicating a ray trace of fluorescence generating light from the source of fluorescence generating light impinging on the retina.

FIG. 17 shows the system of FIG. 16 with a ray trace of fluorescent light from the retina impinging on a fluorescence detector.

FIG. 18 shows the system of FIG. 16 with a ray trace of photoactivating light from a source of photoactivating light targeted to target tissue.

FIG. 19 shows an injection device that is used to deliver photoreactive agent to a specific location of a patient's eye.

FIG. 20 shows the injection device of FIG. 25 being used to deliver photoreactive agent to a specific location of a patient's eye.

FIG. 21 shows a PDT device 2710 that can be used to expose a treated eye region to light.

### DETAILED DESCRIPTION

A. Definitions

Unless defined otherwise, all technical and scientific terms used herein have the same meaning as is commonly understood by one of skill in the art to which this invention belongs. All patents and publications referred to herein are incorporated by reference. In the event that more than one definition is provided herein, the definition in this section controls.

As used herein, photodynamic therapy refers to a therapeutic or diagnostic method involving use of a photoreactive agent and electromagnetic radiation of a sufficient intensity and wavelength to activate the photoreactive agent. The activated photoreactive agent then, through emission of energy, exerts a therapeutic effect, such as destruction of cells or tissue, or allows for diagnosis through detection of the emitted fluorescence energy.

As used herein, a photoreactive agent is a compound or composition that is useful in photodynamic therapy. Such agents are capable of absorbing electromagnetic radiation and emitting energy sufficient to exert a therapeutic effect or sufficient to be detected in diagnostic applications.

As used herein, an activation wavelength range is the wavelength range over which the photoreactive agent is activated.

As used herein, local delivery refers to delivery proximal to the site of administration without substantial delivery to the surrounding tissue or to other tissues of the body.

As used herein, photoreactive light refers to light of sufficient intensity and wavelength to activate the photoreactive agent.

As used herein, fluorescence generating light refers to light of sufficient intensity and wavelength to induce fluorescence of the photoreactive agent.

As used herein, pharmaceutically acceptable derivatives of a compound include salts, esters, enol ethers, enol esters, acetals, ketals, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxy-methyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl ar heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl ar heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein.

As used herein, amelioration of the symptoms of a particular disorder by use of a particular photoreactive agent or pharmaceutical composition thereof in the methods provided herein refers to any lessening, whether permanent or temporary, lasting or transient that can be attributed to or associated with use of the photoreactive agent or pharmaceutical composition thereof in the methods provided herein.

As used herein, a prodrug is a compound that, upon in vivo administration, is metabolized by one more steps or processes or otherwise converted to the biologically, pharmaceutically, diagnostically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

B. Systems and Methods for PDT

Systems and methods for treating neoplastic, neovascular and hypertrophic diseases are provided. In one embodiment, systems and methods for performing photodynamic therapy using localized delivery of a photoreactive agent to target tissue are provided. The photoreactive agent is photoactivated by a non-invasive light source located external to the patient's body.

Photodynamic therapy is a process whereby light is directed to tissues undergoing treatment or investigation that have been rendered photosensitive through the administration of a photoreactive or photosensitizing agent. In certain embodiments, the light is of a specific wavelength, such as the specific wavelength for activation of the photoreactive or photosensitizing agent. The objective of the intervention may be either diagnostic, where the wavelength of light is selected to cause the photoreactive agent to fluoresce, thus yielding information about the tissue without damaging the tissue, or therapeutic, where the wavelength of light delivered to the photosensitive tissue under treatment causes the photoreactive agent to undergo a photochemical interaction with oxygen in the tissue under treatment that yields free radical species, such as singlet oxygen, causing local tissue lysing or destruction.

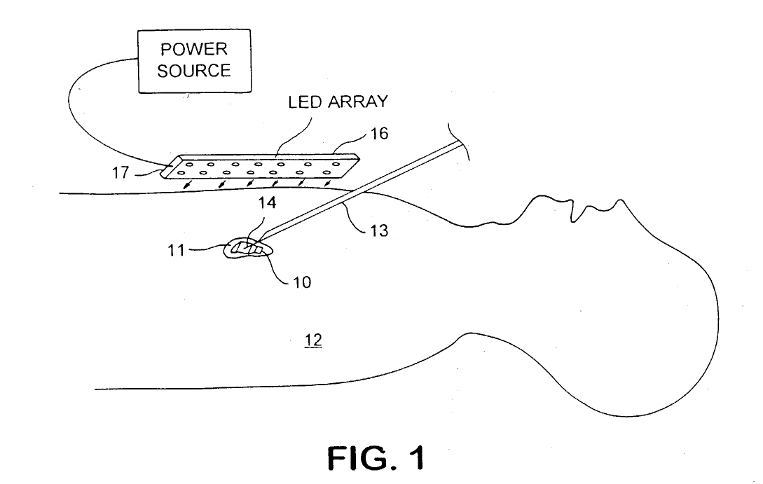

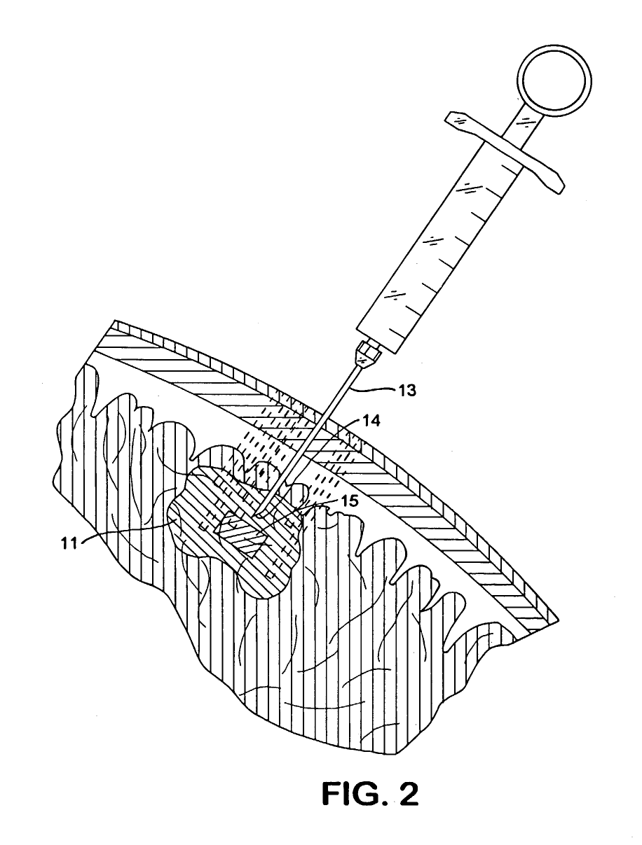

FIGS. 1 and 2 show a photoreactive agent being delivered locally to target tissue 11 of a patient 12. The target tissue 11 of the patient 12 is a tumor located within the chest cavity below a dermal layer of the patient 12. The photoreactive agent 10 is being locally delivered by a hypodermic needle 13 which is inserted into the patient's chest with the tip 14 of the needle disposed within the target tissue 11. Photoreactive agent 10 is being dispensed from the tip 14 of the hypodermic needle 13 and is shown permeating the target tissue 11. FIG. 2 also shows an alternative method and device for local delivery of a photoreactive agent which includes a photoreactive agent depot 15 disposed within the target tissue 11. The photoreactive agent depot 15 is a device that contains photoreactive agent 10 and is configured to dispense the photoreactive agent 10 at a predetermined rate. For some embodiments, the photoreactive agent depot 15 can be a polymer material impregnated with a photoreactive agent 10 that dissolves into the adjacent target tissue 11 over time. Once an appropriate amount of the photoreactive agent 10 has been dispensed into and absorbed by the target tissue 11, the photoreactive agent 10 may then be photoactivated in order to treat the target tissue 11.

The appropriate amount of photoreactive agent 10 to be absorbed by the target tissue will be a factor of the desired clinical result and the specific photoreactive agent 10 used. However, by use of a localized delivery method, as discussed above, less photoreactive agent 10 is used than would be required for the same photoreactive agent 10 delivered intravenously or otherwise systemically to the patient 12.

Once the target tissue 11 has absorbed an appropriate amount of the photoreactive agent 10, a source of electromagnetic radiation 16 having a wavelength within an activation wavelength of the photoreactive agent 10 is used to activate the photoreactive agent 10. A source of electromagnetic radiation 10 consisting of one or more light sources can be used. Various types of light sources can be used, such as, for example, at least one light-emitting diode, laser diode, incandescent light bulb, gas discharge device, polymeric electroluminescent device, halogen bulb, chemical luminescence, vacuum fluorescence, radio frequency excited gas, microwave excited gas, cold cathode fluorescent tube, or combination thereof.

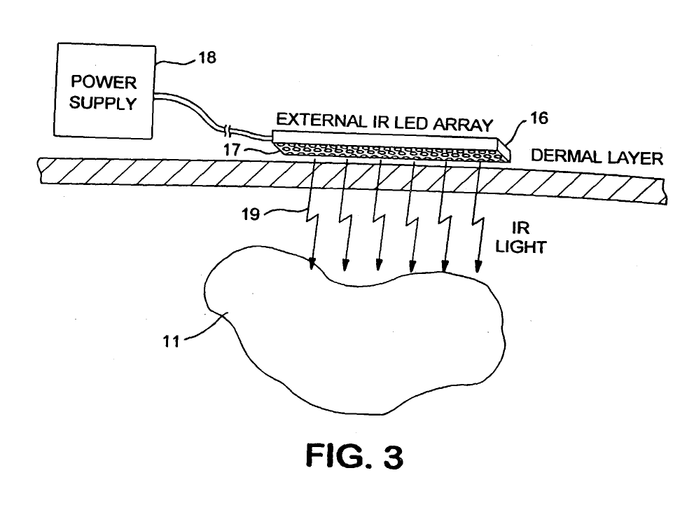

An exemplary source of electromagnetic radiation consisting of an array of light emitting diodes 17 (LEDs) is seen in FIGS. 1 and 3. The LED array 16 can have an emission wavelength of about 500 to about 900, or about 600 to about 700, nanometers, depending on the photoreactive agent used, and is in electrical communication with a power supply unit 18. In some embodiments, long wavelength LEDs 17 can be used that have an emission wavelength of greater than about 700 nanometers in the infrared band up to about 900 nanometers. The light produced by such an array of long wavelength LEDs 17 can easily penetrate tissue and a photoreactive agent 10 having an activation wavelength range corresponding to the long wavelength of the emitted light. The LED array 16 may include LEDs 17 that are made from either polymeric, organic or metallic materials.

The LED array 16 can emit long wavelength infrared light with an output power of about 5 mW/cm² to about 500 mW/cm².

The LED array 16 is shown activated in FIG. 3 with electromagnetic energy in the form of photoreactive light, as shown by the arrows 19, being emitted from the LED array 16 through the dermal layer of the patient 12 and the underlying tissue. The photoactivating light continues to the target tissue 11 and impinges on the photoreactive agent 10 within the target tissue 11. The photoreactive agent 10 then undergoes photochemical excitation and induces formation of a free radical species, such as singlet oxygen, which is toxic to surrounding target tissue 11. The tumor or target tissue 11 is thereby lysed with a minimal amount of photoreactive agent 10 used and without the use of an invasive photoactivation light delivery system such as a fiber optic probe or the like. Because the LED array 16 is external to the patient's body 12, the photoactivating light can be delivered at a rate, which is slower than the rate that would be used if an invasive source of photoactivating light were being used. This results in reduced photobleaching and oxygen consumption, which enhances the efficacy of PDT. In addition, the total dose of light that can be delivered is much greater with an external non-invasive source of photoactivating light 16 because the dose can be administered over a longer period of time as compared with an invasive light source without the risks that are present with an invasive photoactivating light source, such as infection, bleeding and the risks associated with the administration of anesthetics.

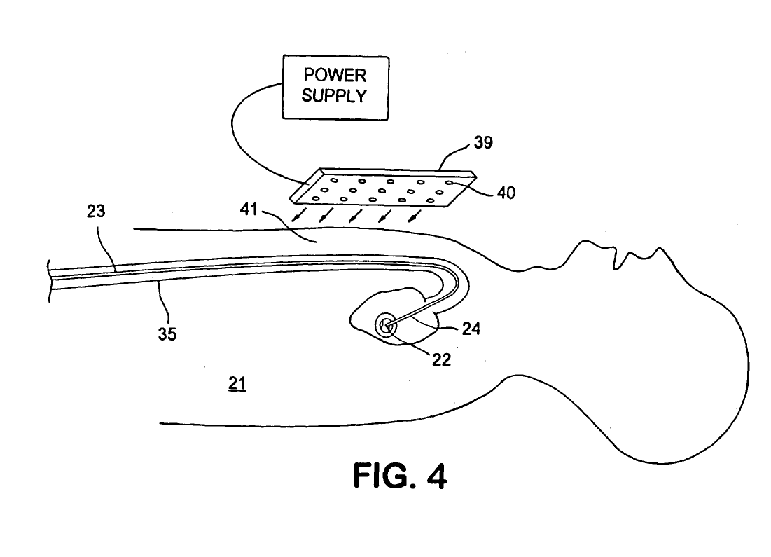

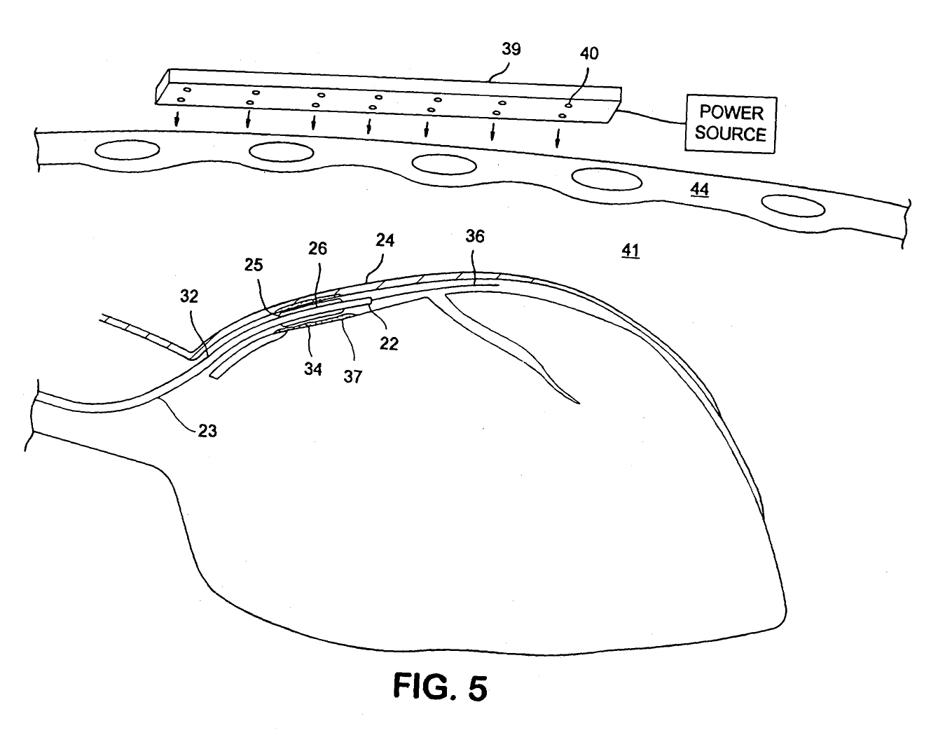

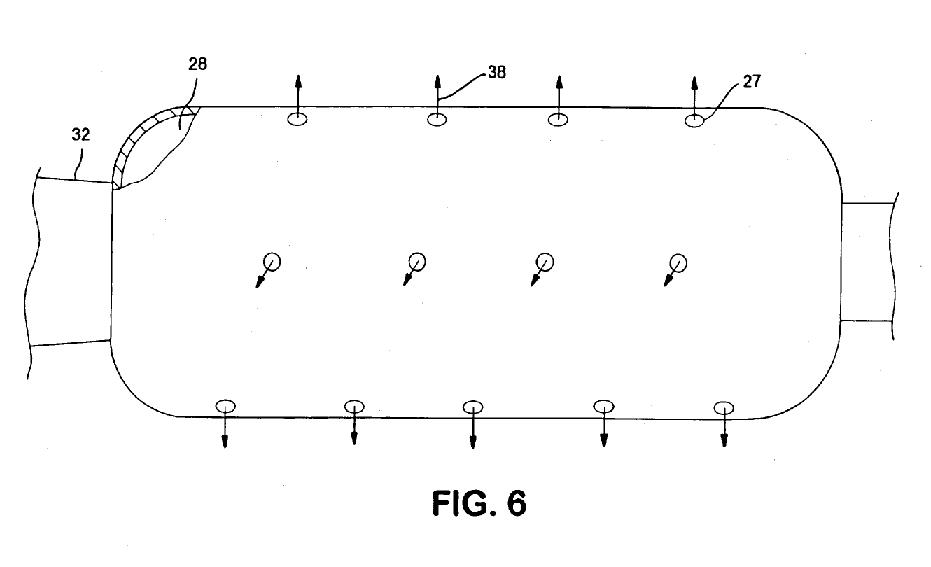

Referring to FIG. 4, a patient 21 is shown with coronary artery disease being treated with PDT. A distal end 22 of a coronary delivery catheter 23 is disposed within a coronary artery 24 of the patient 21 as seen in more detail in FIG. 5. The coronary delivery catheter 23 is a multi-lumen catheter having an optional expandable balloon 25 secured to a distal portion 26 of the catheter 23 and a guidewire lumen (not shown). A plurality of outlet ports 27 are disposed on the expandable balloon 25 as seen more clearly in FIG. 6. The outlet ports 27 are in fluid communication with an interior chamber 28 of the balloon 25, which is in fluid communication with an injection lumen 31 (not shown) disposed within a shaft 32 of the coronary delivery catheter 23. A proximal end 33 of the injection lumen 31 is connected to a Luer adapter at a proximal end (not shown) of the coronary delivery catheter 23 to facilitate injection of a photoreactive agent 34 into the injection lumen 31.

In use, the distal end 22 of the coronary delivery catheter 23 is advanced into the patient's vasculature 35 using a standard percutaneous technique, such as the Seldinger technique. In one embodiment, the coronary delivery catheter 23 is advanced over a coronary guidewire 36 previously placed across the target lesion 37 in the coronary artery 24. The coronary delivery catheter 23 is advanced distally until the expandable balloon 25 is disposed adjacent the target lesion 37. A photoreactive agent 34 is then injected into the injection lumen 31 of the catheter 23 and travels distally in the injection lumen 31 to the interior chamber 28 of the expandable balloon 25, which expands the expandable balloon 25 against the target tissue 37. The photoreactive agent 34 is then expelled from the outlet ports 27, as shown by the arrows 38 in FIG. 6, and into contact with the target tissue 37. This process is continued until the target tissue 37 has absorbed an appropriate amount of the photoreactive agent 34. Thereafter, a source of photoactivating light, such as the LED array 39 shown in FIGS. 4 and 5 can be positioned external to the patient's body 21 adjacent the target tissue 37 and activated.

Upon activation of the LED array 39, photoreactive light having a wavelength within an activation wavelength range of the photoreactive agent 34 travels from the LEDs 40 of the LED array 39 and into the tissue 41 of the patient 21. The photoreactive light passes through the dermal layer 44 of the patient 21 and the underlying tissue 41 until it reaches the target tissue 37 which contains the photoreactive agent 34. The photoreactive agent 34 then undergoes photochemical excitation and induces formation of a free radical species, such as singlet oxygen, which is toxic to surrounding target tissue 37 and the target tissue 37 is destroyed. The coronary catheter delivery catheter 23 can be withdrawn either before or after the administration of photoactivating light, however, it may be desirable to withdraw the catheter 23 prior to administration of the photoactivating light so that the catheter 23 does not prevent any of the photoactivating light from penetrating the target tissue 37. The total dose of photoactivating light that can be delivered is much greater with an external non-invasive source of photoactivating light because the dose can be administered over a long period of time without the risks that would be present with an invasive photoactivating light source, such as infection, bleeding and the risks associated with the administration of anesthetics. In addition, the insertion of an invasive fiber optic photoactivating light source into the patient's vasculature 35 can lead to thrombosis and vessel wall injury including the creation of an intimal flap. These risks are also avoided by use of an external source of photoactivating light.

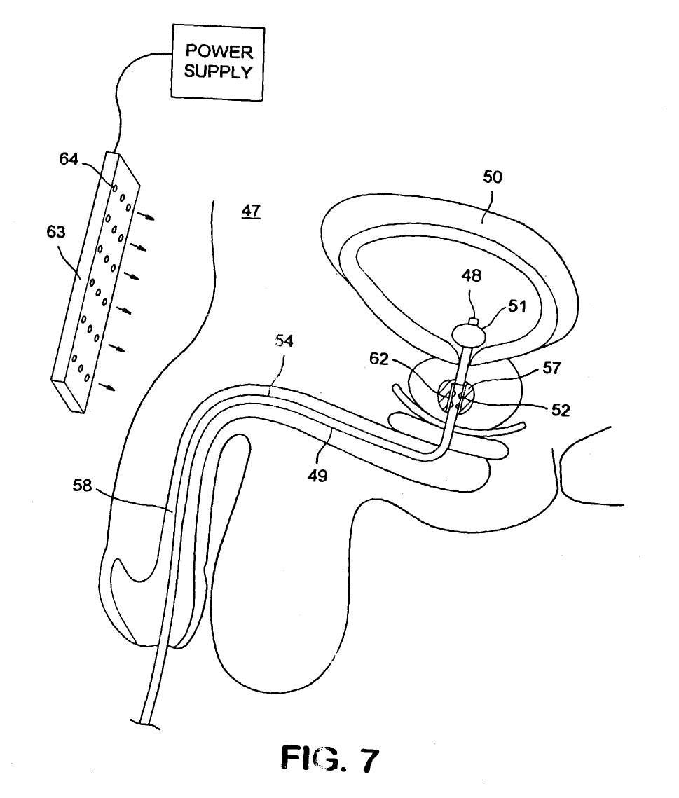

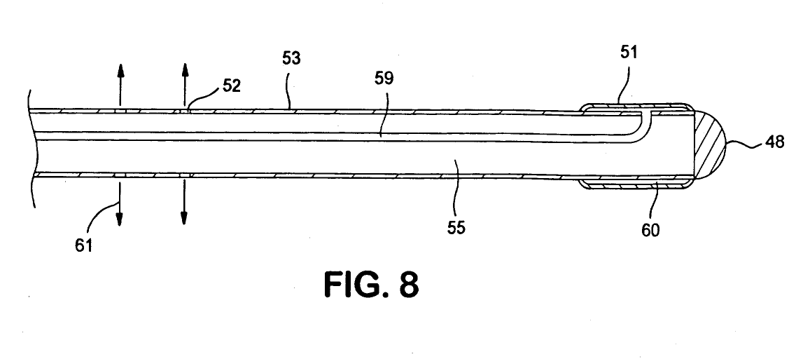

Referring to FIG. 7, a patient 47 is shown with benign prostatic hypertrophy disease being treated with PDT. A distal end 48 of a urinary delivery catheter 49 is disposed within a bladder 50 of the patient 47. The urinary delivery catheter 49 is a multi-lumen catheter having an optional expandable balloon 51 secured to the distal end 48 of the catheter 49. A plurality of outlet ports 52 are disposed in a distal portion 53 of a shaft 54 of the urinary delivery catheter 49 as seen more clearly in FIG. 8. The outlet ports 52 are in fluid communication with a photoreactive agent 10 injection lumen 55 disposed within the shaft 54 of the urinary delivery catheter 49. A proximal end of the photoreactive agent injection lumen is connected to a Luer adapter at a proximal end (not shown) of the urinary delivery catheter 49 to facilitate injection of a photoreactive agent 57 into the injection lumen 55.

In use, the distal end 48 of the urinary delivery 49 catheter is advanced into the patient's urethra 58 using standard techniques. In one embodiment, the urinary delivery catheter 49 will be advanced distally until the expandable balloon 51, in a collapsed state, is disposed within the patient's bladder 50. The expandable balloon 50 can then be expanded by injection of a suitable material, such as saline, into a balloon injection lumen 59 and into an interior chamber 60 of the balloon 51. A photoreactive agent 10 57 is then injected into the photoreactive agent 10 injection lumen 55 of the catheter 49 and travels distally in the injection lumen 55 to the outlet ports 52 and is then expelled from the outlet ports 52, as shown by the arrows 61 in FIG. 8, and into contact with the target tissue 62. This process is continued until the target tissue has absorbed an appropriate amount of the photoreactive agent 10 57. Thereafter, a source of photoactivating light 63, such as the LED array 39 shown in FIGS. 4 and 5 can be positioned external to the patient's body 47 adjacent the target tissue 62 and activated.

Upon activation of the LED array 63, photoreactive light having a wavelength within an activation wavelength range of the photoreactive agent travels from the LEDs 64 of the LED array 63 and into the tissue of the patient 47. The photoreactive light passes through the dermal layer of the patient 47 and the underlying tissue until it reaches the target tissue 62 which contains the photoreactive agent 57. The photoreactive agent 57 then undergoes photochemical excitation and induces formation of a free radical species, such as singlet oxygen, which is toxic to surrounding target tissue 62 and the target tissue 62 is destroyed. The urinary catheter 49 delivery catheter can be withdrawn either before or after the administration of photoactivating light, however, it may be desirable to withdraw the catheter 49 prior to administration of the photoactivating light so that the catheter 49 does not prevent any of the photoactivating light from penetrating the target tissue 62.

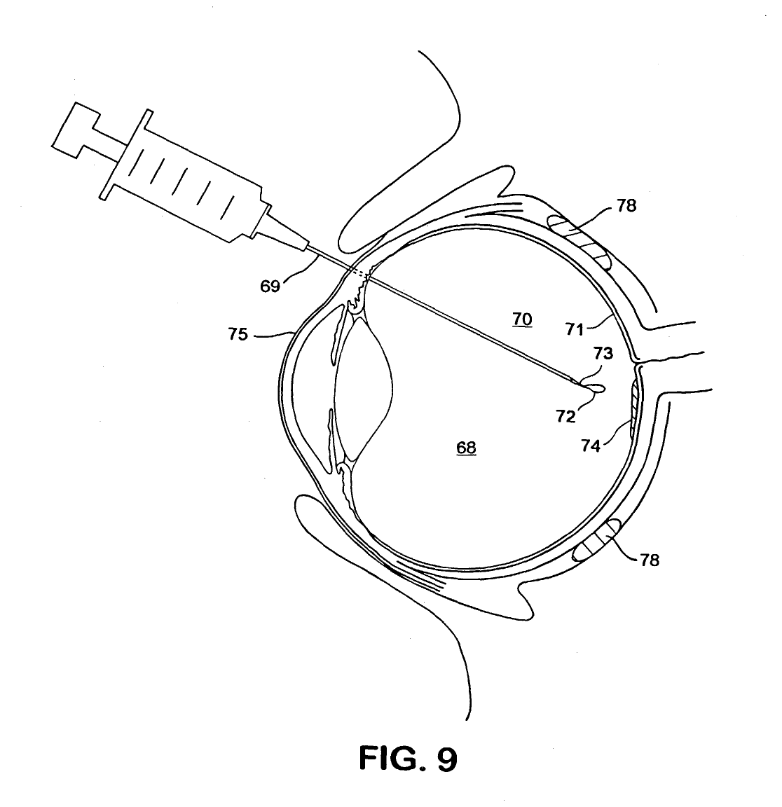

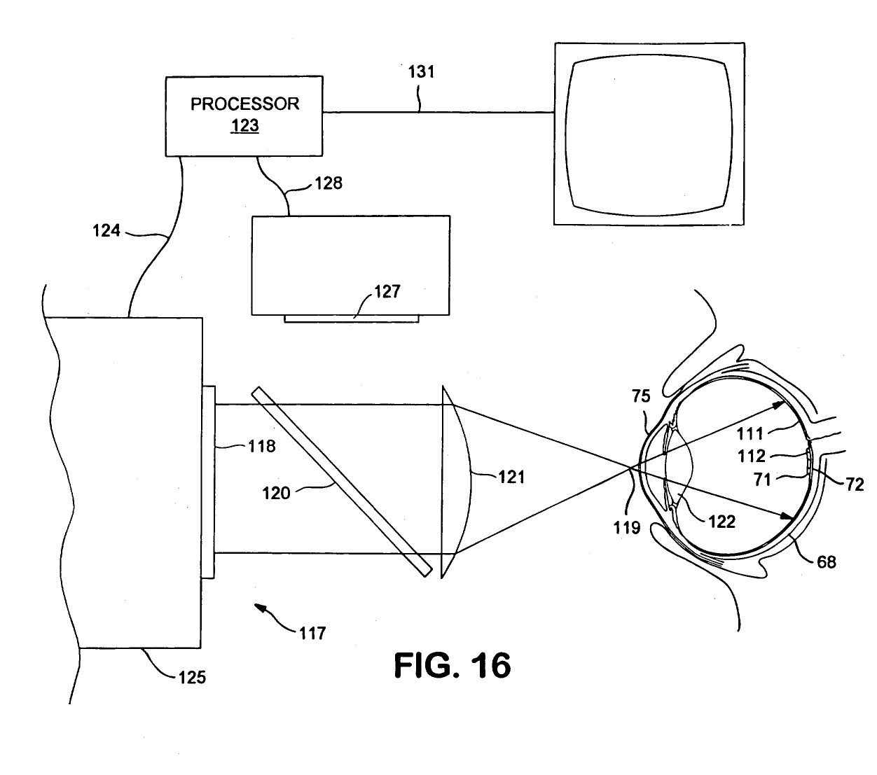

Referring generally to FIGS. 9-18, vascular closure has been observed as one of the consequences of therapeutic PDT which has recently led to the use of PDT in opthalmological disease. The exudative stage of age-related macular degeneration (AMD) with choroidal neovascularization (CNV) commonly leads to rapidly progressive loss of sight. PDT can induce a selective occlusion of CNV via light-induced chemical thrombosis and this effect can be used to effectively treat AMD. Diabetic retinopathy (DR) can be similarly treated. However, destruction of CNV that is not properly limited or targeted to the area requiring treatment can result in undesirable collateral damage to retinal tissue. This, in turn, can lead to reduction in visual acuity. These complications are addressed by a system, such as that shown in FIG. 16, that targets photoactivation energy or light to a desired area of treatment.

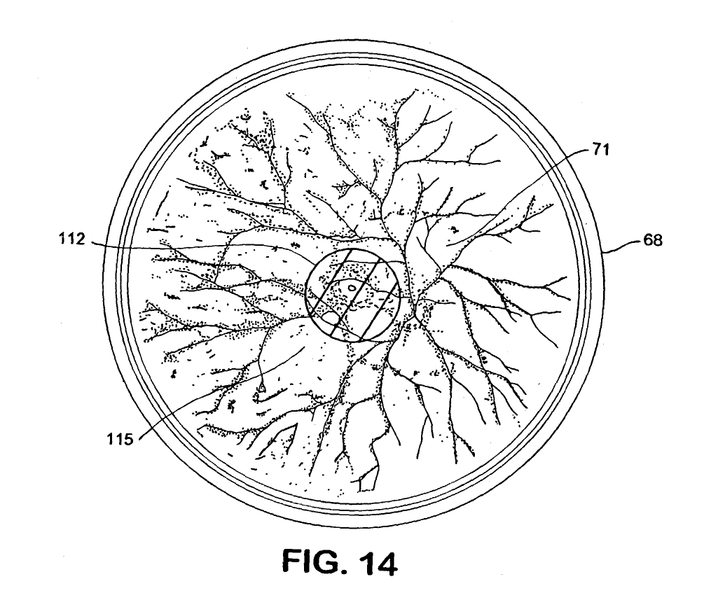

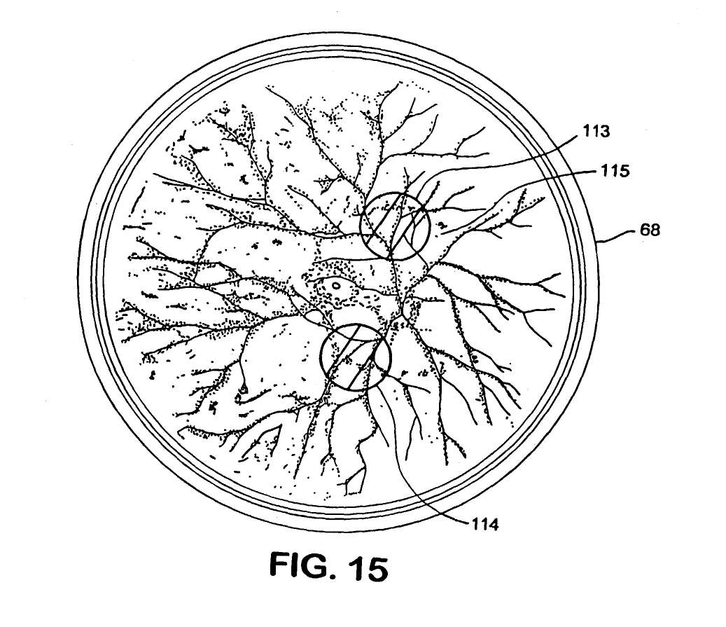

FIG. 9 is a sectional view of a patient's eye 68 being prepared for PDT. A thin hypodermic needle 69 is shown disposed within the vitreous humor 70 of the patient's eye 68 adjacent the retina 71. A photoreactive agent 72 is being dispensed from a distal end 73 of the hypodermic needle 69 adjacent target tissue 74 within the patient's retina 71. In one embodiment of a method of treatment, prior to insertion of the hypodermic needle 69, a corneal surface 75 of the patient's eye 68 is first anesthetized with a topical anesthetic such as Tetracaine® or the like. The hypodermic needle 69 is then advanced into the vitreous 70 and the photoreactive agent 72 injected as a single bolus infusion. It may be necessary is some instances to depress the globe of the eye 68 in order to facilitate posterior placement of the distal end 73 of the hypodermic needle 69 prior to injection of the photoreactive agent 72. The photoreactive agent 72 can be an aqueous formulation that facilitates transport of the drug through the retina 71 and into the target tissue 74, i.e., the neovasculature, shown in FIGS. 14 and 15, beneath the retina 71. The photoreactive agent 72 is thereafter allowed to absorb into the target tissue 74 for a predetermined amount of time. Optionally, the eye 68 may be examined using standard ophthalmic imaging and pressure measurement during this period. Once an appropriate amount of the photoreactive agent 72 has been absorbed by the target tissue 74 in order to achieve the desired clinical result, the photoreactive agent 72 in the target tissue 74 can be photoactivated as discussed below. Note that in the case of “wet” AMD, certain photoreactive agents will be dissipated from the normal retinal tissue after the absorption period and will be localized to the neovessels of the target tissue. For example, a conjugate of a photosensitive agent and an antibody may be used for specific binding to neovessels.

Also shown in FIG. 9 are two photoreactive agent depots 78 which have been placed behind the patient's eye 68 in order to deliver photoreactive agent 72 into the interior structure of the eye 68. The photoreactive agent depots 78 may be made of a polymer material which is impregnated with a suitable photoreactive agent. The polymer can be chosen to allow the photoreactive agent to emanate from the photoreactive agent depots 78 at a predetermined rate. The photoreactive agent 72 then absorbs into the sclera of the patient's eye 68 and eventually perfuses into the target tissue 74 beneath the retina 71.

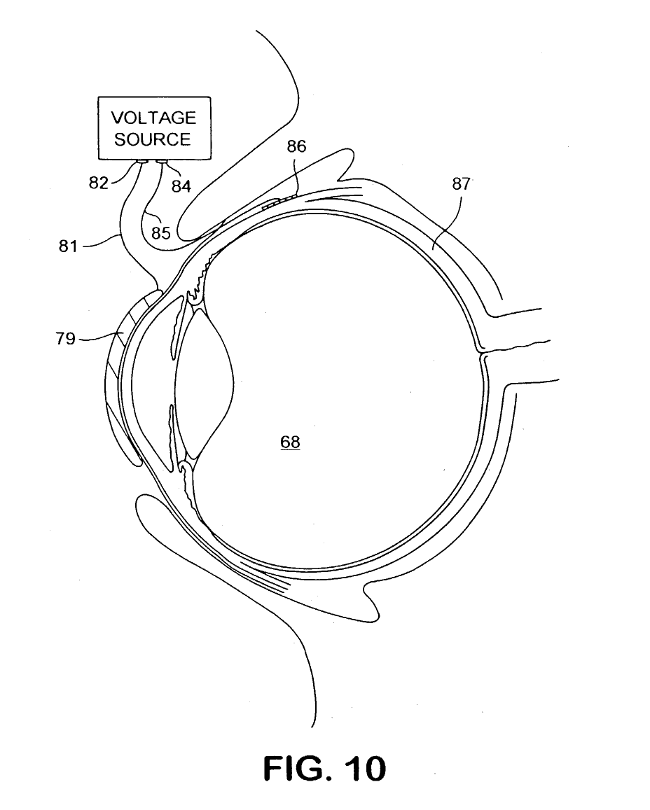

FIG. 10 illustrates an alternative method of localized delivery of a photoreactive agent 72 which includes a contact disk 79 disposed on a corneal surface 75 of the eye 68. The contact disk 79 can have properties similar to those of the photoreactive agent depots 78 discussed above, however, an optional first electrical conductor 81 in electrical communication with the contact disk 79 extends from the contact disk 79 to a first pole 82 of a voltage source 83. A second pole 84 of the voltage source 83 is in electrical communication with a second electrical conductor 85 which is connected to an electrical contact pad 86 in electrical communication with the patient's body, specifically, the sclera 87 of the patient's eye 68. In this way, an electrical voltage potential can be imposed by the voltage source 83 between the contact disk 79 and the corneal surface 75, or any other surface, of the patient's eye 68. The application of such an electrical potential can facilitate perfusion of the photoreactive agent 72 from the contact disk 79 into the patient's eye 68.

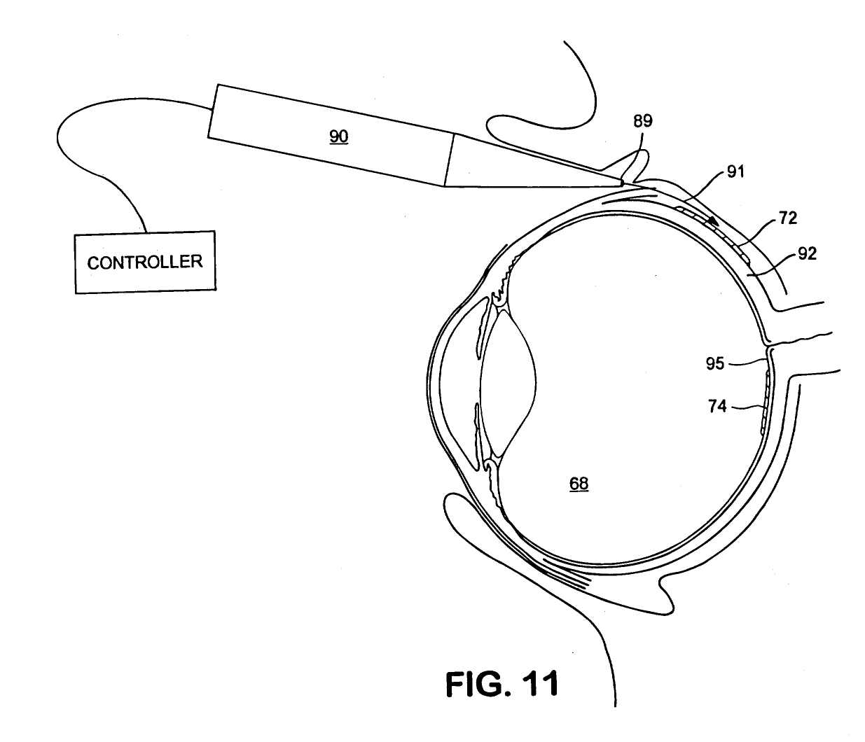

FIG. 11 illustrates another alternative method for localized delivery of a photoreactive agent 72 to target tissue 74 within a patient's eye 68. FIG. 11 is a sectional view of a patient's eye 68 with a distal end 89 of a gas jet injector 90 or drug aerosol device disposed between the eye 68 and eye socket of the patient. Photoreactive agent 72 is delivered by gas jet injection, as shown by the arrows 91 in FIG. 11, to the tissue behind the eye 92 adjacent the target tissue 74 below the patient's retina 71. A controller 93 is shown in electrical communication with the gas jet injector 90 for controlling the duration, pressure, and volume of gas jet injection. By using gas jet injection of the photoreactive agent 72, the photoreactive agent 72 can be distributed to a wide surface area behind the patient's eye 68 which may aid in more rapid transport of the agent 72 to the target tissue 74 within the eye 68. The photoreactive agent 72 can be delivered more posterior in the eye 68 by penetrating the conjunctival membrane 94 with air or another gas during injection which may increase proximity of the photoreactive agent 72 to the macula 95 and posterior retina 71.

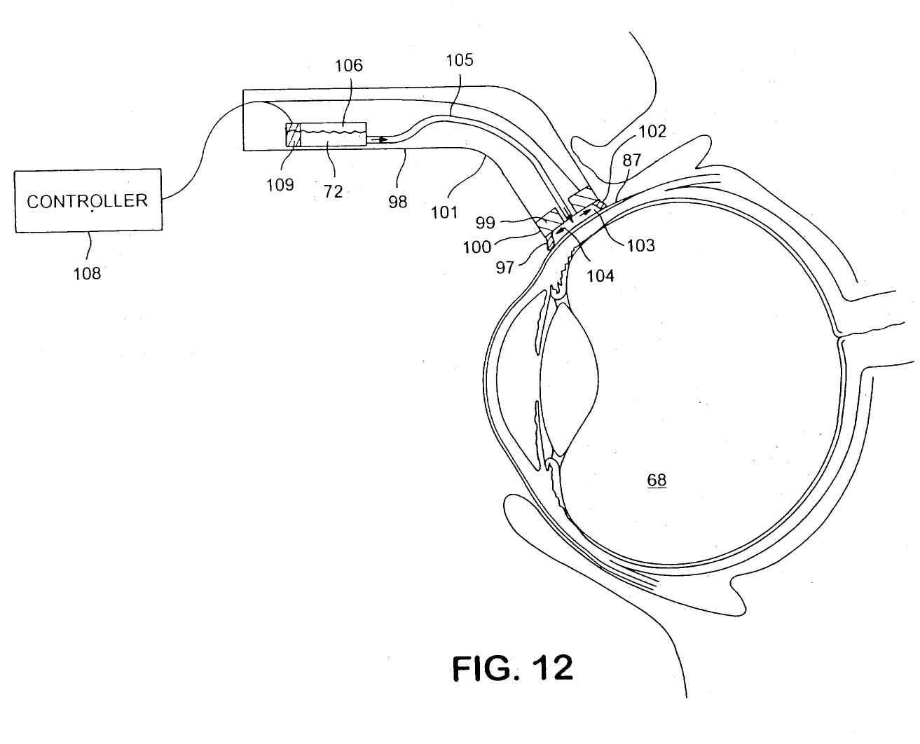

FIG. 12 illustrates yet another embodiment of a device and method for localized delivery of a photoreactive agent 72. FIG. 12 is a sectional view of a patient's eye 68 with a distal end 97 of an ultrasonic probe 98 for delivery of a photoreactive agent 72 disposed on the sclera 87 of the patient's eye 68. The ultrasonic probe 98 includes an ultrasonic emitter 99 disposed in a distal portion 100 of an elongate shaft 101. The ultrasonic emitter 99 generates ultrasonic energy which is transmitted to an outer surface 87 of the patient's eye 68 through a contact ring 102 disposed on a distal end 97 of the elongate shaft 101. The contact ring 102 is in contact with the outer surface 87 of the eye 68 and can form an annular seal between the distal end 97 of the elongate shaft and the outer surface 87 of the eye 68. A distal cavity 103 is disposed within the contact ring 102 which allows for dispersion of a photoreactive agent 72 which is delivered to the distal cavity 103 as shown by the arrows 104 in FIG. 12.

The photoreactive agent 72 is delivered through an injection lumen 105 which is in fluid communication with the distal cavity 103 and a photoreactive agent reservoir 106 disposed in a proximal portion 107 of the elongate probe 98. A controller 108 is in electrical communication with the ultrasonic emitter 99 and a pump 109 disposed within the photoreactive agent reservoir 106. The controller 108 determines the frequency, amplitude and duration of ultrasonic energy produced by the ultrasonic emitter 99. The controller 108 is also configured to control the rate and amount of injection of the photoreactive agent 72 from the photoreactive agent reservoir 106 to the distal cavity 103. Ultrasonic energy is emitted from the ultrasonic emitter 99 once photoreactive agent 72 is disposed within the distal cavity 103 which facilitates permeation of the photoreactive agent 72 into the patient's eye 68 and reduces the time required to deliver an appropriate amount of photoreactive agent 72 to the target tissue 74 within the patient's eye 68. The frequency of the emitted ultrasonic energy can be from about 1 to about 50 MHz, specifically, about 10 to about 40 MHz.

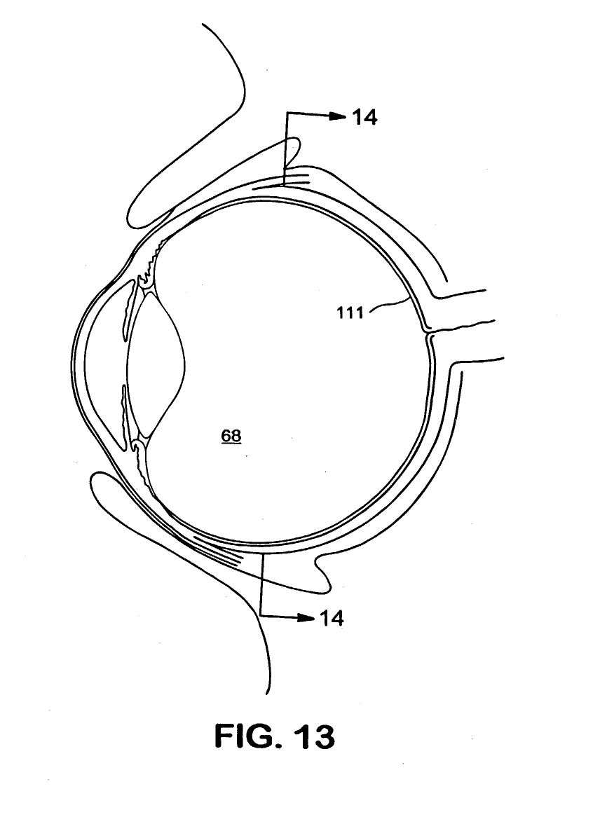

FIG. 13 illustrates a sectional view of a patient's eye 68 that has been dosed with an appropriate amount of photoreactive agent. FIG. 14 is a cross sectional view of the eye 68 of FIG. 13 taken along lines 14-14 in FIG. 13 and illustrates the fundus 111 of the patient's eye 68. In FIG. 14, an area or target tissue 112 is indicated by a hatched area. The target tissue 112 is disposed in an area of the patient's retina 71 that would be consistent with an area of deterioration due to age-related macular degeneration. The target tissue 112 would likely contain neovascularization with the potential for visual loss for the patient. FIG. 15 illustrates a view similar to that of FIG. 14 and shows a first target tissue area 113 and a second target tissue area 114 that would be consistent with areas of deterioration due to diabetic retinopathy. The target tissue areas 112, 113 and 114 of FIGS. 14 and 15 can be dosed with an appropriate amount of photoreactive agent 72 by any of the methods discussed above, as well as other suitable methods. Once the target tissue areas 112, 113 and 114 have been appropriately dosed with a photoreactive agent 72, the photoreactive agent 72 must be photoactivated. Indiscriminate photoactivation of the photoreactive agent 72 in the tissue of the patient's eye can be undesirable because of the possible risk of damage to healthy collateral tissue 115 adjacent the target tissue areas 112, 113 and 114. A system for performing PDT 117 such as shown in FIG. 16 can be useful for avoiding such risks.

The PDT system 117 shown in FIG. 16 includes a source of fluorescence generating light 118 which is configured to illuminate the fundus 111 of a patient's eye 68 as indicated by the ray trace arrows 119 in FIG. 16. The fluorescence generating light is emitted by the source of fluorescence generating light 118 and travels through a beam splitting member 120, a focusing member 121 and the cornea 75 and lens 122 of the patient's eye 68. The fluorescence generating light then impinges on the retina 71 of the patient's eye 68 and the tissue underlying the retina 71 and has sufficient intensity and wavelength to cause fluorescence of a photoreactive agent 72 without causing photoactivation of the photoreactive agent 72. The target tissue areas 112, 113 and 114, and any other tissue that contain a concentration of photoreactive agent 72 will then fluoresce.

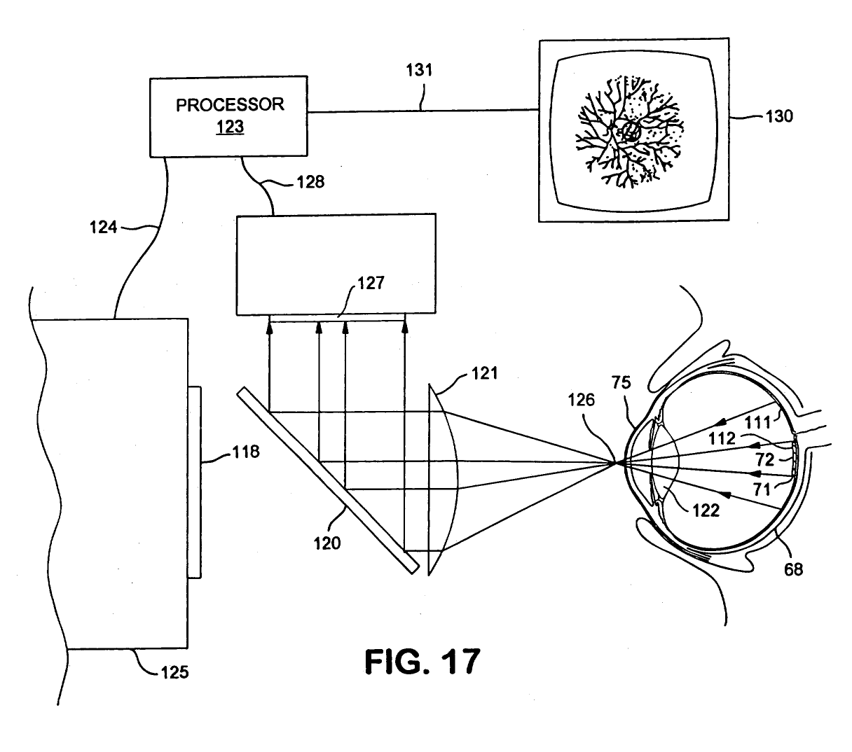

Initiation of emission of the fluorescence generating light from the source of fluorescence generating light 118 is carried out by a processor 123 which is in electrical communication with the source of fluorescence generating light 118 with a bundle of electrical conductors 124. In one embodiment, the source of fluorescence generating light 118 includes a laser 125 having an operating wavelength of about 600 to about 700 nanometers, specifically, about 660 to about 670 nanometers. The beam splitting member 120 can be any of a suitable variety of commercially available beam splitters which is relatively transmissive in the direction of the fluorescence generating light shown in FIG. 16 and relatively reflective for light traveling in the opposite direction, as shown in FIG. 17. The focusing member 121 can be a commercially available lens made from any suitable material which is transmissive for the wavelength of the fluorescence generating light.

Once the fluorescence generating light hits the target tissue 112, 113 and 114 and surrounding tissue 115, and the photoreactive agent 72 therein fluoresces, the fluorescent light then travels from the target tissue 112, 113 and 114 and surrounding tissue 115 out of the patient's eye 68 and back into the focusing member 121 as shown in FIG. 17 by the arrows 126. After passing through the focusing member 121, the fluorescent light hits the beam splitter member 120 and substantially reflects up to a fluorescence detector 127 which is configured to measure the intensity of fluorescent light emanating from each coordinate point of the fundus 111 of the patient's eye 68. The fluorescence detector 127 can be a charged couple chip or device, but could also use slit lamp photography in order to plot the fluorescence distribution. The time course of the photography will be determined by the initial fluorescence appearance and distribution in the choroid and later in the retina.

This fluorescence response data is then captured by the processor 123 which is in electrical communication with the fluorescence detector 127 with a bundle of electrical conductors 128. The processor 123 then analyzes the fluorescence response data and generates a virtual map that indicates the coordinates of the target tissue 112, 113 and 114 relative to the surrounding normal tissue 115 as indicated by the ray trace arrows in FIG. 17. In some embodiments, the target tissue 112, 113 and 114 is distinguished from the surrounding tissue 115 by the presence of supra-threshold photoreactive agent 72 concentrations in the tissue. The processor 123 may also display the virtual map, or any other fluorescence response data visually on an optional monitor display 130 which is in electrical communication with the processor 123 with a bundle of electrical conductors 131.

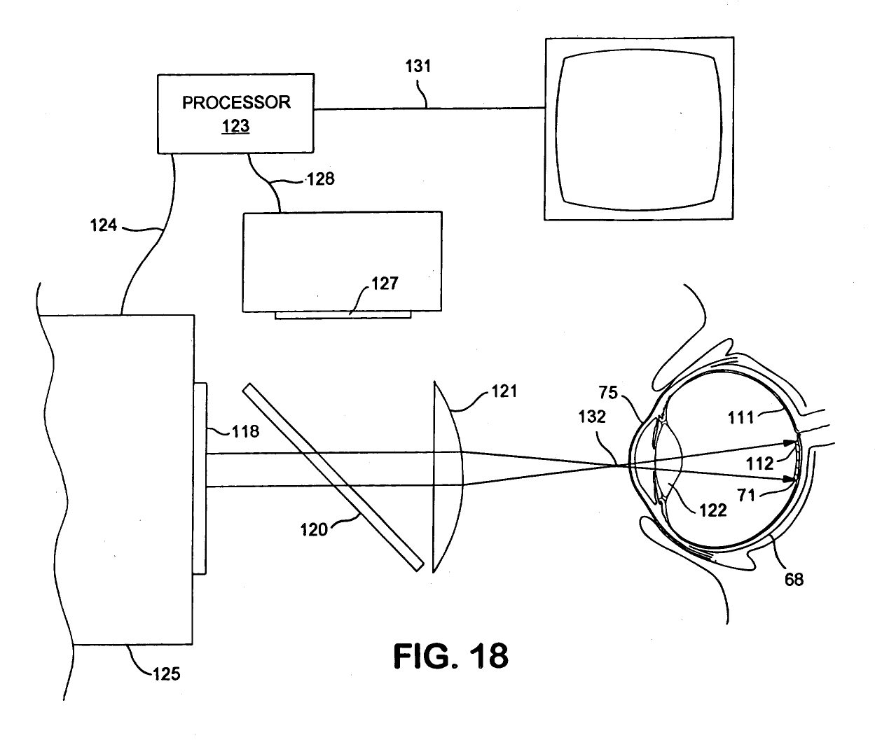

Once the processor 123 has generated a virtual map which distinguishes the coordinates of the target tissue 112, 113 and 114 from the surrounding normal or non-target tissue 115, the processor 123 can then be used to control the output beam of a source of photoactivating light 118 so that the photoactivating light is directed only to the target tissue area 112, 113 and 114 of the patient's eye 68 as shown in FIG. 18 by the ray trace arrows 132. The source of photoactivating light 118 can be the same laser 125 as that used for the source of fluorescence generating light 118, or another device can be used. The controller 123 can control the delivery of the photoactivating light by any suitable method including aiming and scanning a thin beam of photoactivating light across the entire region of target tissue 112, 113 and 114 while avoiding the collateral areas 115 of healthy tissue. In this way, only the photoreactive agent 72 within the target tissue areas 112,113 and 114 are photoactivated with the production and lysing effect of singlet oxygen or the like.

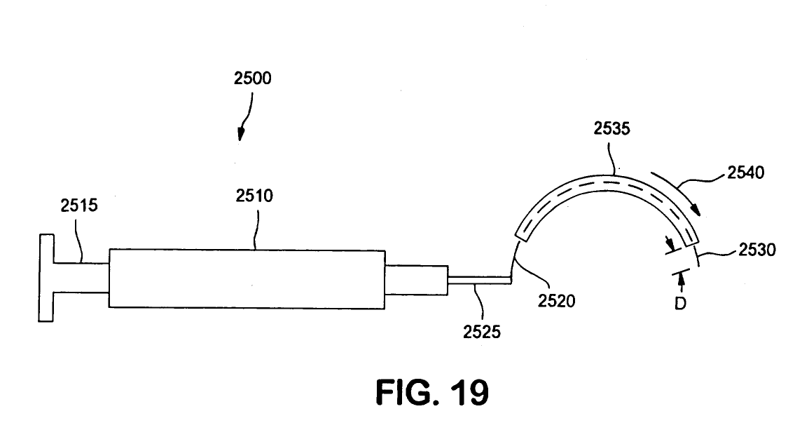

FIG. 19 shows yet another device that can be used for localized delivery of a photoreactive agent. The device is an injection device 2500 that can be used for localized delivery of a photoreactive agent to a patient's eye. The injection device 2500 includes a syringe 2510 in which is mounted a plunger 2515 that is movably mounted in the syringe 2510. A hypodermic needle 2520 is coupled to the syringe by a flexible coupling 2525. The needle has a sharpened distal tip 2530 that can be used to penetrate eye tissue. A cannula or sheath 2535 covers at least a portion of the needle 2520. The needle 2520 and the sheath 2535 both have a curved shape that can conform to the curvature of the outer surface of a patient's eye. Thus, the needle and sheath define a substantially circular curvature, although the curvature can vary. The curvature of the needle/sheath can vary based upon the curvature of the eye with which the device will be used. In one embodiment, the needle and sheath conform to a radius of approximately 12 mm. As described below, the curved shape of the needle/sheath facilitate placement of the distal tip 2530 of the needle 2520 to posterior regions of the eye. The needle and sheath can be manufactured of a variety of materials, including stainless steel and plastic.

The needle 2520 can be retractable with respect to the sheath 2535 such that the distal tip 2530 can be retracted so that it is positioned within the sheath 2535. The needle can also be advanced in a distal direction (represented by the arrow 2540 in FIG. 19) such that the distal tip 2530 protrudes outwardly from the sheath 2535, such as is shown in FIG. 19. In one embodiment, the needle 2520 can only be advanced a limited distance so that the distal tip 2530 can only extend a distance D outward from the edge of the sheath 2535. This feature can prevent inadvertent over-penetration of the needle into the eye tissue.

As mentioned, a flexible coupling 2525 attaches the needle 2520 to the syringe. The flexible coupling 2525 permits the curved needle 2520 to be moved to various orientations relative to the syringe 2510 in order to facilitate positioning of the needle relative to the eye upon delivery of the photoreactive agent. The syringe can be filled with a desired photoreactive agent, which can be dispensed out of the distal tip 2530 of the needle 2520 by pressing the plunger 2515 in a well-known manner.

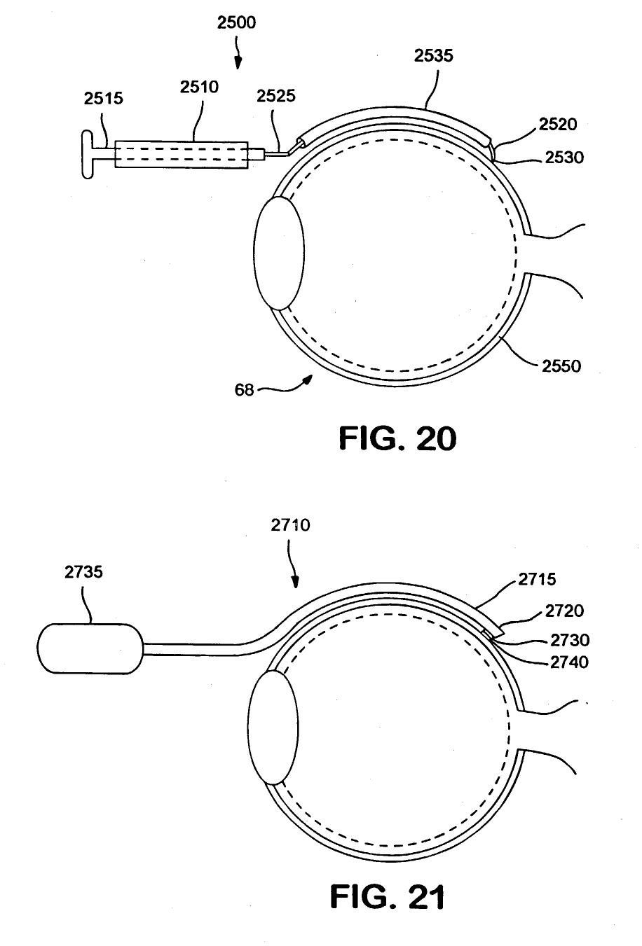

A method of using the injection device is now described with reference to FIG. 20, which shows a sectional view of a patient's eye 68. Various anatomical details of the eye 68 are omitted from FIG. 20 for clarity of illustration. In use, the needle 2520 and sheath 2535 are inserted between the eye and the eye socket (not shown) such that the needle and sheath are positioned substantially adjacent the outer surface of the eye 68. The curved shape of the needle and sheath facilitate such insertion. In one embodiment, the needle 2520 is retracted into the sheath 2535 prior to placement of the needle around the eye. Thus, the sharpened, distal tip 2530 of the needle 2520 is positioned within the sheath 2535 while the needle and sheath are inserted around the eye. In this manner, the sheath 2535 will shield the sharpened, distal tip 2530 of the needle 2520 from contact with the eye and thereby eliminate the risk of the sharp needle injuring the eye while the needle is being positioned. The distal edge of the sheath 2535 can have an atraumatic shape in order to reduce the risk of the sheath damaging the eye.

When the distal tip of the needle 2520 is at a desired location relative to the eye 26, the needle is then advanced so that the distal tip 2530 protrudes from the sheath 2535. The needle 2520 is of sufficient length so that the distal tip can reach any desired location of the eye, such as diseased tissue comprised of the neovascular membrane (not shown). The distal tip can then be advanced so that it penetrates the eye to a desired depth. In one embodiment, the needle penetrates only the sclera 2550 of the eye 68 without penetrating any deeper. It should be appreciated, however, that the needle can optionally penetrate the eye to any desired depth. When the needle has penetrated the eye 68 to the desired depth, the photoreactive agent is delivered to target region of the eye by dispensing the photoreactive agent through the distal tip of the needle 2520. As mentioned, this is accomplished by pressing the plunger 2515 so that the agent is forced out of the distal tip of the needle 2515 and into the eye 68.

After the photoreactive agent has been delivered to the target region of the eye, the target region can be exposed to photoreactive light to thereby photoactivate the agent. FIG. 21 shows a PDT device 2710 that can be used to expose a treated eye region to light. The PDT device 2710 includes an elongated arm 2715 that has a curved shape. The curvature of the arm 2715 conforms to the curvature of the outer surface of a patient's eye. This facilitates positioning of the arm 2715 around the outer surface of the eye. The curvature of the arm 2715 can vary based upon the curvature of the eye with which the device will be used. In one embodiment, the arm 2715 follows a curve with a radius of approximately 12 mm.

The arm 2715 has a distal end 2720 upon which is mounted a source of photoreactive light. The source of light can be, for example, an LED 2730. The LED 2730 is positioned such that it can emit light in a predetermined direction, such as toward a target region of the eye. The LED 2730 is electrically coupled to a source of power (not shown) and a controller 2735 that can be used to control power to the LED 2730. A lens 2740 can be positioned over the LED 2730 in order to focus the light from the LED 2730. The lens 2740 can be manufactured of any suitable material, such as, for example, Polymethyl methacrylate (PMMA).

In use, the PDT device 2710 is deployed such that the distal tip 2720 is positioned adjacent the region of the eye to be treated. The device 2710 is oriented so that the LED 2730 is positioned to emit light toward the target region of the eye. As mentioned, the curvature of the elongated arm 2715 facilitates positioning of the arm 2715 around the outer surface of the eye. Once the LED is properly positioned, the LED is activated so that it emits light toward the region of the eye that has been treated with the photoreactive agent.

C. Photoreactive Agents

Any chemical compound that absorbs light may be used in the methods provided herein (see, e.g., Kreimer-Birnbaum (1989) Sem. Hematol. 26:157-173). Photoreactive agents for use in the methods provided herein include, but are not limited to, indocyanine green, toluidine blue, prodrugs such as aminolevulinic acid, texaphyrins, benzoporphyrins, phenothiazines, phthalocyanines, porphyrins, merocyanines, psoralens, protoporphyrin, methylene blue, Rose Bengal (see, e.g., Picaud et al. (1990) Brain Res. 531:117-126 and Picaud et al. (1993) J. Neurosci. Res. 35:629-642), chlorins such as mono-L-aspartyl chlorin e6, alkyl ether analogs of chlorins, purpurins, bacteriochlorins, pheophorbides, pyropheophorbides, cationic dyes and any other agent that absorbs light in a range of about 500 to about 1100 nanometers. Photoreactive agents for use in the methods provided herein are also disclosed in commonly assigned U.S. patent applications, Ser. No. 09/078,329, filed May 13, 1998, entitled “Controlled Activation of Targeted Radionuclides”, Ser. No. 60/116,234, filed Jan. 15, 1999, entitled “Targeted Transcutaneous Cancer Therapy”, Ser. No. 09/271,575, filed Mar. 18, 1999, entitled “Targeted Transcutaneous Cancer Therapy”, Ser. No. 09/905,501, filed Jul. 13, 2001, entitled “Targeted Transcutaneous Cancer Therapy”, Ser. No. 09/905,777, filed Jul. 13, 2001, entitled “Non-invasive Vascular Therapy”, Ser. No. 60/175,689, filed on Jan. 12, 2000, entitled “Novel Treatment for Eye Disease”, Ser. No. 09/760,362, filed on Jan. 12, 2001, entitled “Novel Treatment for Eye Disease”, and Ser. No. 60/116,235, filed on Jan. 15, 1999, entitled “Non-invasive Vascular Therapy”, the disclosure of each of which is hereby incorporated by reference in its entirety. Photoreactive agents for use in the methods provided herein are also disclosed in U.S. Pat. Nos. 6,319,273, RE37,180, 4,675,338, 4,693,885, 4,656,186, 5,066,274, 6,042,603, 5,913,884, 4,997,639, 5,298,018, 5,308,861, 5,368,841, 5,952,366, 5,430,051, 5,567,409, 5,942,534, and U.S. patent application Publication No. 2001/0,022,970. In one embodiment, the photoreactive agent for use in the methods provided herein is taporfin sodium, also referred to as mono-L-aspartyl chlorin e6, (+)-tetrasodium (2S,3S)-18-carboxylato-20-[N—(S)-1,2-dicarboxylatoethyl]carbamoylmethyl-13-ethyl-3,7,12,17-tetramethyl-8-vinylchlorin-2-propanoate, NPe6 or ME2906.

In another embodiment, the photoreactive reagents for use in the methods provided herein include but are not limited to porphyrins such as PHOTOPHRIN™ (a QLT, Ltd. brand of sodium porfimer), and FOSCAN™, which is a brand of chlorin.

In another embodiment, the photoreactive reagents for use in the methods provided herein include but are not limited to indocyanine green (ICG), methylene blue, touidine blue, aminolevulinic acid (ALA), chlorins, phthalocyanines, porphyrins, pupurins, texaphyrins, and other photosensitizer agents that have characteristic light absorption peaks in a range of from about 500 nm to about 1100 nm.

In another embodiment, the photoreactive reagents for use in the methods provided herein include but are not limited to chlorins, bacteriochlorins, phthalocyanines, porphyrins, purpurins, merocyanines, psoralens, benzoporphyrin derivatives (BPD), and porfimer sodium and pro-drugs such as delta-aminolevulinic acid, which can produce photosensitive agents such as protoporlphyrin IX, and other suitable photosensitive compounds including ICG, methylene blue, toluidine blue, texaphyrins, and any other agent that absorbs light in a range of 500 nm to 1100 nm.

In another embodiment, the photoreactive reagents for use in the methods provided herein include but are not limited to LUTRIN™ (lutetium texaphyrin, brand; Pharmacyclics, Inc. Sunnyvale, Calif.), and bacteriochlorphylls.

In another embodiment, the photoreactive reagents for use in the methods provided herein include but are not limited to clorins, bacteriochlorphylls, phthalocyanines, porphyrins, purpurins, merocyanines, psoralens, benzoporphyrin derivatives (BPD) and porfimer sodium and pro-drugs such as delta-aminolemulinic acid, which can produce drugs such as protoporphyrin; and others such as indocyanince green (ICG); methylene blue; toluidine blue; texaphyrins; pyroheohorbide compounds; bacteriochlorphyll derivatives; alkyl ether analogs of chlorins, and an other agent that absorbs light in a range of 500 nm to 1100 nm.

In another embodiment, the photoreactive reagents for use in the methods provided herein include but are not limited to PURYLITIN™ (tin ethyl etiopurpurin) or VERTEPORFIN™ (a liposomal benzoporphyrin derivative).

In another embodiment, the photoreactive reagents for use in the methods provided herein include but are not limited to photosensitizers selected from:

1. Photofrin®.

2. Synthetic diporphyrins and dichlorins

3. Hydroporphyrins, e.g., chlorins and bacteriochlorins of the tetra(hydroxyphenyl) porphyrin series

4. phthalocyanines

5. O-substituted tetraphenyl porphyrins (picket fence porphyrins)

6. 3,1-meso tetrakis (o-propionamido phenyl) porphyrin

7. Verdins

8. Purpurins, e.g., tin and zinc derivatives of octaethylpurpurin (NT2), and etiopurpurin (ET2)

9. Chlorins, e.g., chlorin e6, and mono-I-aspartyl derivative of chlorin e6

10. Benzoporphyrin derivatives (BPD), e.g., benzoporphyrin monoacid derivatives, tetracyanoethylene adducts of benzoporphyrin, dimethyl acetylenedicarboxylate adducts of benzoporphyrin, Diels-Adler adducts, and monoacid ring “a” derivative of benzoporphyrin

11. Low density lipoprotein mediated localization parameters similar to those observed with hematoporphyrin derivative (HPD)

12. sulfonated aluminum phthalocyanine (Pc) sulfonated AIPc disulfonated (AIPcS.sub.2) tetrasulfonated derivative sulfonated aluminum naphthalocyanines chloroaluminum sulfonated phthalocyanine (CASP)

13. zinc naphthalocyanines

14. anthracenediones

15. anthrapyrazoles

16. aminoanthraquinone

17. phenoxazine dyes

18. phenothiazine derivatives

19. chalcogenapyrylium dyes cationic selena and tellurapyrylium derivatives

20. ring-substituted cationic PC

21. pheophorbide alpha.

22. hematoporphyrin (HP)

23. protoporphyrin

24. 5-amino levulinic acid

In another embodiment, the photoreactive reagents for use in the methods provided herein include but are not limited to photosensitizers selected from members of the following classes of compounds: porphyrins, chlorins, bacteriochlorins, purpurins, phthalocyanines, naphthalocyanines, texaphyrines, and non-tetrapyrrole photosensitizers. Specific examples are Photofrin™, benzoporphyrin derivative, tin etiopurpurin, sulfonated chloroaluminum phthalocyanine and methylene blue.

In another embodiment, the photoreactive reagents for use in the methods provided herein include but are not limited to BPD which is a second generation porphyrin photosensitizer that diffuses rapidly from microvasculature and disseminates throughout a joint. In addition, BPD has a low affinity for chondrocytes and articular cartilage following systemic or intra-articular injection. CASPc, a phthalocyanine inactivates growth factors TGF-.beta. and bFGF.

In another embodiment, the photoreactive reagents for use in the methods provided herein include but are not limited to photosensitizers selected from:

1. Photofrin®.

2. Synthetic diporphyrins and dichlorins

3. Hydroporphyrins such as chlorins and bacteriochlorins of the tetra(hydroxyphenyl) porphyrin series

4. phthalocyanines (PC) with or without metal substituents, e.g., chloroaluminum phthalocyanine (CASP) with or without varying substituents

5. O-substituted tetraphenyl porphyrins (picket fence porphyrins) p1 6. 3,1-meso tetrakis (o-propionamido phenyl) porphyrin

7. Verdins

8. Purpurins tin and zinc derivatives of octaethylpurpurin (NT2) etiopurpurin (ET2)

9. Chlorins/chlorin e6 mono-l-aspartyl derivative of chlorin e6 di-l-aspartyl derivative of chlorin e6

10. Benzoporphyrin derivatives (BPD) benzoporphyrin monoacid derivatives tetracyanoethylene adducts of benzoporphyrin dimethyl acetylenedicarboxylate adducts of benzoporphyrin Diels-Adler adducts monoacid ring “a” derivative of benzoporphyrin

11. sulfonated aluminum PC sulfonated AIPc disulfonated (AIPcS.sub.2) tetrasulfonated derivative sulfonated aluminum naphthalocyanines

12. naphthalocyanines with or without metal substituents with or without varying substituents

13. anthracenediones

14. anthrapyrazoles

15. aminoanthraquinone

16. phenoxazine dyes

17. phenothiazine derivatives

18. chalcogenapyrylium dyes cationic selena and tellurapyrylium derivatives

19. ring-substituted cationic PC

20. pheophorbide derivative

21. hematoporphyrin (HP)

22. other naturally occurring porphyrins

23. 5-aminolevulinic acid and other endogenous metabolic precursors

24. benzonaphthoporphyrazines

25. cationic imminium salts

26. tetracyclines

In another embodiment, the photoreactive reagents for use in the methods provided herein include but are not limited to compounds of the formula (I):

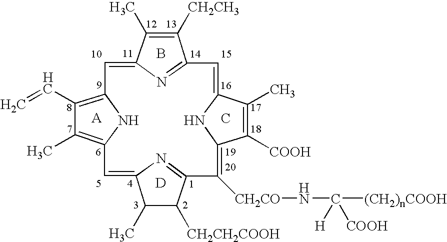

where n stands for an integer of 1 or 2, or a pharmaceutically acceptable salt thereof; and a pharmaceutically acceptable carrier for the effective ingredient.

In another embodiment, the photoreactive agent has the general formula

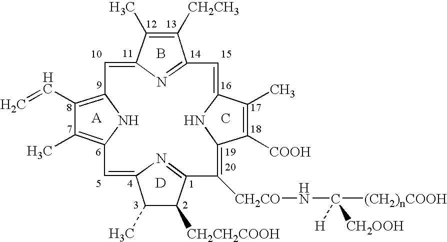

Among the compounds of the general formula shown above, the compound where n is 1 is such compound wherein L-aspartic acid is combined via an amido linkage with the side chain group CH₂COOH at the 20-position. This particular compound is mono-L-aspartyl-chlorin e6. This mono-L-aspartyl-chlorin e6 may be in the form of its tetra-sodium salt at the four carboxyl groups of the compound.

Among the compounds of the general formula shown above, the compound where n is 2 is such compound wherein L-glutamic acid, in stead of said L-aspartic acid, is combined via the amido linkage of the side chain group CH₂COOH at the 20-position of the tetrapyrrole ring. This compound is mono-L-glutamyl-chlorin e6.

In another embodiment, the photoreactive reagents for use in the methods provided herein include but are not limited to compounds of the formula:

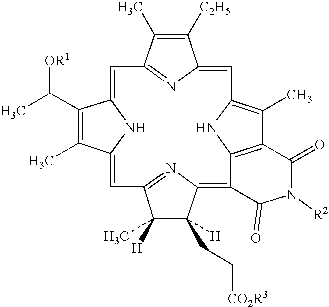

where R¹, R² and R³ are independently alkyl of 3 through about 10 carbon atoms; provided that, R¹.and R² together contain at least six carbon atoms. R³ is preferably methyl or ethyl and R² and R³ are preferably alkyl of 3 through 8 carbon atoms.

In another embodiment, the photoreactive reagents for use in the methods provided herein include but are not limited to the following classes: purpurins, verdins, chlorins, phthalocyanines, phorbides, bacterioschlorophylls, porphyrins, chalcogenapyryliums, texaphyrins, xanthenes, benzophenoxazines, phenothiazines, di- and triayl methanes, and kryptocyanines. Exemplary members of the above classes are listed in the following Table.

| Class | Exemplary Compound |
|---|---|
| Purpurins | Tin Ethyl Etiopurpurin |
| Verdins | Coproverdin-II-tripotassium Salt |
| Chlorins | Octaethyl Chlorin |
| Phthalocyanines | Chloaluminum Sulfonated Phthalocyanine |
| Phorbides | Mono-L-Aspartyl Chlorin e6 |
| Bacteriochlorophylls | Bacteriochlorophyll-a |
| Porthyrins | Protoporphyrin-IX |
| Chalcogenapyryliums | Chalcogenapyrylium 8b |
| Texaphyrins | Texaphyrin |
| Xanthenes | Rhodamine 123 |
| Benzophenoxazines | Nile Blue |
| Phenothiazines | Methylene Blue |
| Di- and Triayl Methanes | Victoria Blue-BO |
| Kryptocyanines | EDKC |

*EDKC = N,Nbis[2 ethyl1,3-dioxolane] kryptocyanine

In another embodiment, the photoreactive reagents for use in the methods provided herein include but are not limited to the halogenated xanthanes below:

Fluorescein

4′,5′-Dichlorofluorescein

2′,7′-Dichlorofluorescein

4,5,6,7-Tetrachlorofluorescein

2′,4′,5′,7′-Tetrachlorofluorescein

Dibromofluorescein

Solvent Red 72

Diiodofluorescein

Eosin B

Eosin Y

Ethyl Eosin

Erythrosin B

Phloxine B

Rose Bengal

Rose Bengal Lithium Salt

Rose Bengal Derivative I

Rose Bengal Derivative II

4,5,6,7-Tetrabromoerythrosin

In another embodiment, the photoreactive reagents for use in the methods provided herein include but are not limited to psoralen and its derivatives (including 5-methoxypsoralen [or 5-MOP]; 8-methoxypsoralen [8-MOP]; 4,5′,8-trimethylpsoralen [TMP]; 4′-aminomethyl-4,5′,8-trimethylpsoralen [AMT]; 4′-hydroxymethyl-4,5′,8-trimethylpsoralen [HMT]; 5-chloromethyl-8-methoxypsoralen, Angelicin [isopsoralen]; 5-methlyangelicin [5-MIP]; and 3-carbethoxypsoralen); various porphyrin and hematoporphyrin derivatives (including haematoporphyrin derivative [HPD]; Photofrin II; benzoporphyrin derivative [BPD]; protoporphyrin IX [Pp IX]; dye hematoporphyrin ether [DHE]; polyhematoporphyrin esters [PHE]; 13,17-N,N,N-dimethylethylethanolamine ester of protoporphyrin [PH1008]; tetra(3-hydroxyphenyl)porphyrin [3-THPP]; tetraphenylporphyrin monosulfonate [TPPS1]; tetraphenylporphyrin disulfonate [TPPS2a]; dihematoporphyrin ether; meso-tetraphenyl-porphyrin; and mesotetra(4N-methylpyridyl)porphyrin [T4MPyP]) along with various tetraazaporphyrins (including octa-(4-tert-butylphenyl)-tetrapyrazinoporphyrazine [OPTP]; tetra-(4-ten-butyl)phthalocyanine [t.sub.4-PcH.sub.2]; and tetra(4-tert-butyl) phthalocyanatomagnesium [t.sub.4-PcMg]); various phthalocyanine derivatives (including chloroaluminum-sulfonated phthalocyanine [CASPc]; chloroaluminum phthalocyanine tetrasulfate [AIPcTS]; mono-, di-, tri- and tetra-sulphonated aluminum phthalocyanines [including AISPc, AIS2Pc, AIS3Pc and AIS4Pc]; silicon phthalocyanine [SiPc IV]; zinc(II) phthalocyanine [ZnPc]; bis(di-isobutyl octadecylsiloxy)silicon 2,3-naphthalocyanine [isoBOSINC]); and Ge(IV)-octabutoxy-phthalocyanine various rhodamine derivatives (including rhodamine-101 [Rh-101]; rhodamine-110 [Rh-110]; rhodamine-123 [Rh-123]; rhodamine-19 [Rh-19]; rhodamine-560 [Rh-560]; rhodaine-575 [Rh-575]; rhodamine-590 [Rh-590]; rhodamine-610 [Rh-610]; rhodamine-640 [Rh-640]; rhodamine-6 G [Rh-6G]; rhodamine-700 [Rh-700]; rhodamine-800 [Rh-800]; rhodamine-B [Rh—B]; sulforhodamine 640 or 101; and sulforhodamine B); various coumarin derivatives (including coumarin 1, 2, 4, 6, 6H, 7, 30, 47, 102, 106, 120, 151, 152, 152A, 153, 311, 307, 314, 334, 337, 343, 440, 450, 456, 460, 461, 466, 478, 480, 481, 485, 490, 500, 503, 504, 510, 515, 519, 521, 522, 523, 535, 540, 540A, 548); various benzophenoxazine derivatives (including 5-ethylamino-9-diethylamimobenzo[a]-phenoxazinium [EtNBA]; 5-ethylamino-9-diethylaminobenzo[a]phenothiaziniuna [NBS]; and 5-ethylamino-9-iethylaminobenzo[a]phenoselenazinium [EtNBSe]); chlorpromazine and its derivatives; various chlorophyll and bacteriochlorophyU derivatives (including bacteriochlorin a [BCA]); various metal-ligand complexes, such as tris(2,2′-bipyridine)ruthenium (II) dichloride (RuBPY); pheophorbide a [Pheo a]; merocyanine 540 [MC 540]; Vitamin D; 5-amino-laevulinic acid [ALA]; photosan; chlorin e6, chlorin e6 ethylenediamide, and mono-L-aspartyl chlorin e6; pheophorbide-a [Ph-a]; phenoxazine Nile blue derivatives (including various phenoxazine dyes); various charge transfer and rediative transfer agents, such as stilbene, stilbene derivatives and 4-(N-(2-hydroxyethyl)-N-methyl)-aminophenyl)-4′-(6-hydroxyhexylsulfonyl )stilbene (APSS).

In certain embodiments, the photoreactive agents for use in the methods provided herein are aminocarboxylic acid adducts of a tetrapyrrole containing atleast three carboxyl groups. In other embodiment, the compounds are di or tetrahydrotetrapyrrole carboxylic acids. In other embodiment, the compounds are pharmaceutically acceptable salts of the of the carboxylic acids such as salts of alkali metals, alkaline earth metals, ammonium and amines.

In another embodiment, the aminocarboxylic acids are amino monocarboxylic acids selected from serine, glycine, α-aminoalanine, β-aminoalanine, ε-amino-n-caproic acid, piperidine-2-carboxylic acid, piperidine-6-carboxylic acid, pyrrole-2-carboxylic acid, piperidine-2-propionic acid, pyrrole-5-acetic acid, and similar such acids. In other embodiment, the amino acids are the naturally occurring .alpha.-amino monocarboxylic acids such as serine, alanine or glycine.

In another embodiment, the amino carboxylic acids are dicarboxylic acids selected from α-aminosuccinic acid (aspartic acid), α-aminoglutaric acid (glutamic acid), β-aminoglutaric acid, β-aminosebacic acid, 2,6-piperidine dicarboxylic acid, 2,5-pyrrole dicarboxylic acid, 2-carboxypyrrole-5-acetic acid, 2-carboxypiperidine 6-propionic acid, α-aminoadipic acid, and α-aminoazelaic acid. In other embodiment, the amino dicarboxylic acids are the naturally occurring α-amino dicarboxylic acids such as aspartic acid and glutamic acid.

In another embodiment, the compounds are mono-, di- or polyamides of amino monocarboxylic acid and a tetrapyrrole containing atleast three carboxyl groups of the formula:

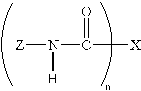

wherein Z is the aminomonocarboxylic acid residue less the amino group and X is the tetrapyrrole residue less the carboxy group and “n” is an integer from 1 to 4.

In another embodiment, the compounds are fluorescent mono- or polyamides of an aminocarboxylic acid and tetrapyrrole compound of the formula:

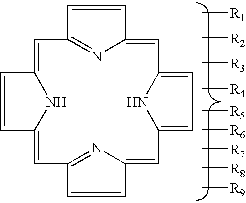

or the corresponding di- or tetrahydrotetrapyrroles, wherein

R₁ is methyl;

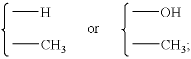

R₂ is H, vinyl, ethyl, —CH(OH)CH₃, acetyl, —C(H)═O, —CH₂CH₂CO₂H, ═CHCHO or

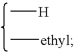

R₃ is methyl,

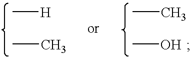

R₄ is H, vinyl, ethyl, —CH(OH)CH₃, —CH₂CH₂CO₂H, ═CHCHO or

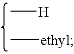

R₅ is methyl;

R₆ is H, —CH₂CH₂CO₂H, —CH₂CH₂CO₂R, or —COOH;

R₇ is —CH₂CH₂CO₂H, —CH₂CH₂CO₂R, or

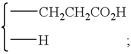

R₈ is methyl,

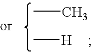

R₉ is H, —COOH, —CH₂COOH or methyl; provided that when R₁, R₂, R₃, R₄, R₇ and R₈ represent two substituents or are divalent and attached to the same carbon, the respective pyrrole ring to which they are attached, is a dihydropyrrole;

R is lower alkyl or benzyl;

R₆ and R₉ taken together are

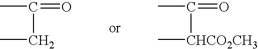

with the proviso that at least one of R₁-R₉ includes a free carboxyl group; and salts thereof.

In another embodiment the compounds are derived from tetrapyrroles of the formula:

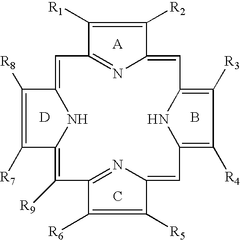

or the corresponding di- or tetrahydrotetrapyrroles and salts thereof, wherein R₁-R₉ are as previously defined.

In another embodiment the photoreactive agents are compounds of the formula:

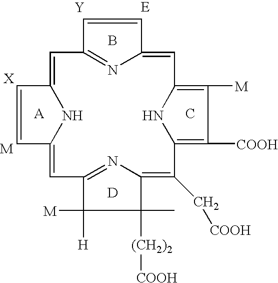

wherein,

X═H, vinyl, ethyl, acetyl or formyl;

Y=methyl, formyl or

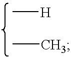

M=methyl; and

E=ethyl or

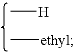

and pharmaceutically-acceptable salts thereof.

In another embodiment, X, Y, M and E are as defined above with the proviso that the compound is not chlorin e6.

In another embodiment X is H, vinyl, ethyl, acetyl or formyl; Y is methyl or formyl; M is methyl; and E is ethyl.

In another embodiment, the photoreactive agents are selected from coproporphyrin III, deuteroporphyrin IX, hematoporphyrin IX, protoporphyrin IX, photoprotoporphyrin IX, mesoporphyrin IX, pyropheophorbide a, transmesochlorin IX, pheophorbide a, chlorine e₄, chlorine e₆, mesochlorin e₄, isochlorin e₄, mesoisochlorin e₄, mesochlorin e₆, bacteriopheophorbide a, pyrobacteriopheophorbide a,_bacteriochlorin e₆, bacteriochlorin e₄, bacterioisochlorin e₄, bacteriochlorin e₆, 2-desvinylchlorin e₆ (or deuterochlorin e₆), 2-acetylchlorin e₆, 2-formylchlorin e₆ and rhodin g₇.

In another embodiment, the photoreactive agents are selected from coproporphyrin III, deuteroporphyrin IX, hematoporphyrin IX, protoporphyrin IX, photoprotoporphyrin IX, mesoporphyrin IX, pyropheophorbide a, transmesochlorin IX, pheophorbide a, chlorine e₄, chlorine e₆, mesochlorin e₄, isochlorin e₄, mesoisochlorin e₄, mesochlorin e₆, bacteriopheophorbide a, pyrobacteriopheophorbide a, bacteriochlorin e₆, bacteriochlorin e₄ and bacterioisochlorin e₄.

In another embodiment, the photoreactive agents are selected from chlorine e6, mesochlorin e₆, bacteriochlorin e₆, 2-desvinylchlorin e₆ (or deuterochlorin e₆), 2-acetylchlorin e₆, 2-formylchlorin e₆ and rhodin g₇.

In another embodiment, the photoreactive agents are chlorin derivatives selected from mono, di and triserinyl chlorin e₆; mono, di and triserinyl mesochlorin e₆; mono, di and trithreoninyl chlorin e₆; mono, di and trithreoninyl chlorin e₆; mono, di and triglycyl acetylchlorin e₆; mono, di and triserinyl rhodin g₇; mono, di and trimethionyl formylchlorin e₆; mono, di and trithreoninyl rhodin g₇; mono, di and tricysteinyl chlorin e₆; and mono, di and tricysteinyl rhodin g₇.

In another embodiment, the compounds are chlorine derivatives selected from mono and diaspartyl trans-mesochlorin IX; mono and diglutamyl trans-mesochlorin IX; mono, di and triaspartyl chlorin e₆; mono, di and triaspartyl mesochlorin e₆; mono, di and triglutamyl chlorin e₆; mono, di and triglutamyl mesochlorin e₆; mono and diaspartyl chlorin e₄; mono and diaspartyl mesochlorin e₄; mono and diaspartyl isochlorin e₄; mono and diaspartyl mesochlorin e₄; mono and diglutamyl chlorin e₄; mono and diglutamyl mesochlorin e₄; mono and diglutamyl isochlorin e₄; mono and diglutamyl mesoisochlorin e₄; monoaspartylpyropheophorbide a; monoglutamylpyropheophorbide a; monoaspartylpheophorbide a; monoglutamylpheophorbide a; mono and diaspartylphotoprotoporphyrin IX; mono and diglutamylphotoprotoporphyrin IX and mono and di-L-alpha-aminoadipyl trans-mesochlorin IX.

In another embodiment, the compounds are chlorine derivatives selected from mono, di and triaspartyl chlorin e₆; mono, di and triaspartyl mesochlorin e₆; mono, di and triglutamyl chlorin e₆; mono, di and triglutamyl mesochlorin e₆; moni, di and triaspartyl acetylchlorin e₆; mono, di and triaspartyl rhodin g₇; mono, di and triaspartyl formylchlorin e₆; mono, di and triglutamyl rhodin g₇; mono, di and triglutamyl acetylchlorin e₆; mono, di and triglutamyl acetylchlorin e₆; mono, di and triglutamyl formylchlorin e₆; mono, di and triaspartyl deuterochlorin e₆; and mono, di and triglutamyl deuterochlorin e₆.

In another embodiment, the photoreactive agents are bacteriochlorine derivatives selected from mono, di and triserinyl bacteriochlorin e₆; mono, di and trithreoninyl bacteriochlorin e₆; and mono, di and tricysteinyl bacteriochlorin e₆.

In another embodiment, the compounds are bacteriochlorin derivatives selected from mono and diaspartylbacteriochlorin e₄; mono and diglutamylbacteriochlorin e₄; mono and diaspartylbacterioisochlorin e₄; mono and diglutamylbacterioisochlorin e₄; mono, di and triaspartylbacteriochlorin e₆; mono, di and triglutamylbacteriochlorin e₆; monoaspartylpyrobacteriopheophorbide a; monoglutamylpyrobacteriopheophorbide a; monoaspartylbacteriopheophorbide a; and monoglutamylbacteriopheophorbide a.

In another embodiment, the compounds are bacteriochlorin derivatives selected from mono, di and triaspartyl bacteriochlorin e₆ and mono, di and triglutamyl bacteriochlorin e₆.

In another embodiment, the compounds are porphyrin derivatives selected from mono and diaspartylmesoporphyrin IX; mono and diglutamylmesoporphyrin IX; mono and diaspartylprotoporphyrin IX; mono and diglutamyl protoporphyrin IX; mono and diaspartyldeuteroporphyrin IX; mono and diglutamyldeuteroporphyrin IX; mono, di, tri and tetraaspartylcoproporphyrin III (isomer mixture); mono, di, tri and tetraglutamylcoporphyrin III; mono and diaspartylhematoporphyrin IX and mono and diglutamylhematoporphyin IX.

D. Preparation of the Photoreactive Agents

The photoreactive agents for use in the methods provided herein may be prepared from readily available starting materials by methods well known to those of skill in the art, or routine modification thereof, or are commercially available (e.g., from Sigma-Aldrich Chemical Co., Milwaukee, Wis.). Methods for preparation of the photoreactive agents are disclosed in commonly assigned U.S. patent applications, Ser. No. 09/078,329, filed May 13, 1998, entitled “Controlled Activation of Targeted Radionuclides”, Ser. No. 60/116,234, filed Jan. 15, 1999, entitled “Targeted Transcutaneous Cancer Therapy”, Ser. No. 09/271,575, filed Mar. 18,1999, entitled “Targeted Transcutaneous Cancer Therapy”, Ser. No. 09/905,501, filed Jul. 13, 2001, entitled “Targeted Transcutaneous Cancer Therapy”, Ser. No. 09/905,777, filed Jul. 13, 2001, entitled “Non-invasive Vascular Therapy”, Ser. No. 60/175,689, filed on Jan. 12, 2000, entitled “Novel Treatment for Eye Disease”, Ser. No. 09/760,362, filed on Jan. 12, 2001, entitled “Novel Treatment for Eye Disease”, and Ser. No. 60/116,235, filed on Jan. 15, 1999, entitled “Non-invasive Vascular Therapy”, the disclosure of each of which is hereby incorporated by reference in its entirety. Methods for preparation of the photoreactive agents for use in the methods provided herein are also disclosed in, e.g., U.S. Pat. Nos. 6,319,273, RE37,180, 4,675,338, 4,693,885, 4,656,186, 5,066,274, 6,042,603, 5,913,884, 4,997,639, 5,298,018, 5,308,861, 5,368,841, 5,952,366, 5,430,051, 5,567,409, 5,942,534, and U.S. patent application Publication No. 2001/0,022,970. Methods for the preparation of taporfin sodium, also known as mono-L-aspartyl chlorin e6 are disclosed in, e.g., U.S. Pat. Nos. RE37,180, 4,675,338 and 4,693,885.

E. Formulation of Pharmaceutical Compositions

The photoreactive agents for use in the methods provided herein may be formulated as pharmaceutical compositions prior to local administration. The pharmaceutical compositions contain a therapeutically or diagnostically effective amount of a photoreactive agent that is useful in photodynamic therapy. The compositions contain one or more photoreactive agents, in one embodiment one photoreactive agent. Typically the photoreactive agents described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more photoreactive agents or pharmaceutically acceptable derivatives is (are) mixed with a suitable pharmaceutical carrier or vehicle. The photoreactive agents may be derivatized as the corresponding salts, esters, enol ethers or esters, acids, bases, solvates, hydrates or prodrugs prior to formulation, as described above. The concentrations of the photoreactive agents in the compositions are effective for delivery of an amount, upon administration, that is useful for photodynamic therapy, such as in the methods provided herein.

Typically, the compositions are formulated for single dosage administration. To formulate a composition, the weight fraction of photoreactive agent is dissolved, suspended, dispersed or otherwise mixed in a selected vehicle at an effective concentration such that the treated condition is relieved or ameliorated. Pharmaceutical carriers or vehicles suitable for administration of the photoreactive compounds provided herein include any such carriers known to those skilled in the art to be suitable for the particular mode of administration.

In addition, the photoreactive agents may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulted compound, pelleted by centrifugation, and then resuspended in PBS.

The photoreactive agent is included in the pharmaceutically acceptable carrier in an amount sufficient to exert a therapeutically or diagnostically useful effect in the absence of undesirable side effects on the patient treated. The therapeutically or diagnostically effective concentration may be determined empirically by testing the compounds in vitro and in vivo systems well known to those of skill in the art and then extrapolated therefrom for dosages for humans.

The concentration of photoreactive agent in the pharmaceutical composition will depend on absorption, inactivation and excretion rates of the photoreactive agent, the physicochemical characteristics of the agent, the dosage schedule, and amount administered as well as other factors known to those-of skill in the art. For example, the amount that is delivered is sufficient to exert a photodynamic therapeutic or diagnostic effect, as described herein.

Typically a therapeutically effective dosage should produce a tissue concentration of photoreactive agent of from about 0.1 ng/cm³ to about 50-100 μg/cm³. The pharmaceutical compositions typically should provide a dosage of from about 0.001 mg to about 2000 mg of photoreactive agent. Pharmaceutical dosage unit forms are prepared to provide from about 1 mg to about 1000 mg and preferably from about 10 to about 500 mg of the photoreactive agent or a combination of photoreactive agents per dosage unit form.

The photoreactive agent may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the compositions.

Pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the photoreactive agents described herein or pharmaceutically acceptable derivatives thereof are mixed with a suitable pharmaceutical carrier or vehicle for local administration to form pharmaceutical compositions. Photoreactive agents are included in an amount effective for ameliorating one or more symptoms of, or for treating or preventing diseases or disorders via photodynamic therapy or diagnosis, as described herein.

The compositions are intended to be administered locally. Solutions or suspensions used for parenteral, intradermal or subcutaneous application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the photoreactive agents exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the photoreactive agents, such as prodrugs of the compounds may also be used in formulating effective pharmaceutical compositions.

Upon mixing or addition of the photoreactive agent(s), the resulting mixture may be a solution, suspension, emulsion or the like. The form of the resulting mixture depends upon a number of factors, including the intended mode of administration and the solubility of the photoreactive agent in the selected carrier or vehicle. The effective concentration is sufficient for ameliorating the symptoms of the disease, disorder or condition treated or is sufficient for diagnostic applications, and may be empirically determined.

The pharmaceutical compositions are provided for administration to humans and animals in unit dosage forms, such as sterile parenteral solutions or suspensions, containing suitable quantities of the photoreactive agents or pharmaceutically acceptable derivatives thereof. The pharmaceutically therapeutically or diagnostically active photoreactive agents and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically or diagnostically active compound sufficient to produce the desired therapeutic or diagnostic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject or to be useful is diagnostic applications.

Dosage forms or compositions containing photoreactive agent in the range of 0.005% to 100% with the balance made up from non-toxic carrier may be prepared. The contemplated compositions may contain 0.001%-100% active ingredient, preferably 0.1-85%, typically 75-95%.

The photoreactive agents or pharmaceutically acceptable derivatives may be prepared with carriers that protect the compound against rapid elimination from the body, such as time release formulations or coatings. The compositions may include other active compounds to obtain desired combinations of properties. The photoreactive agents, or pharmaceutically acceptable derivatives thereof as described herein, may also be advantageously administered for therapeutic or prophylactic purposes together with another pharmacological agent known in the general art to be of value in treating one or more of the diseases or medical conditions referred to herein. It is to be understood that such combination therapy constitutes a further aspect of the methods of treatment and diagnosis provided herein.

1. Injectables, Solutions and Emulsions

Local parenteral administration, generally characterized by injection, either subcutaneously, intramuscularly or intravenously is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. Briefly, a photoreactive agent is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The photoreactive agent diffuses through the outer polymeric membrane in a release rate controlling step. The percentage of photoreactive agent contained in such parenteral compositions is highly dependent on the specific nature thereof, as well as the activity of the compound and the needs of the subject.

Parenteral administration of the compositions includes local subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the photoreactive agent is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Injectables are designed for local administration. Typically a therapeutically effective dosage is formulated to contain a concentration of at least about 0.1% w/w up to about 90% w/w or more, preferably more than 1% w/w of the photoreactive agent to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

2. Articles of Manufacture

The photoreactive agents or pharmaceutically acceptable derivatives may be packaged as articles of manufacture containing packaging material, a photoreactive agent or pharmaceutically acceptable derivative thereof, which is effective for photodynamic therapy or diagnosis, within the packaging material, and a label that indicates that the photoreactive agent, or pharmaceutically acceptable derivative thereof, is used for photodynamic therapy or diagnosis.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the photoreactive agents provided herein are contemplated as are a variety of treatments for any disease or disorder in which photodynamic therapy or diagnosis is indicated.

Since modifications will be apparent to those of skill in this art, it is intended that this invention be limited only by the scope of the appended claims.

What is claimed is:

1. A method of performing photodynamic therapy on a patient comprising:
a) locally delivering a photoreactive agent having an activation wavelength range to target tissue of a patient; and
b) photoactivating the photoreactive agent of the target tissue with electromagnetic radiation having a wavelength within the activation wavelength range that travels from outside the patient's body to the target tissue within the patient's body.

2. The method of claim 1, further comprising allowing the target tissue to absorb a clinically beneficial amount of the photoreactive agent prior to step b) and after step a).

3. The method of claim 1, wherein the photoreactive agent is locally delivered to the target tissue by injection with a hypodermic needle and further comprising advancing the hypodermic needle through the patient's body to the target tissue within the patient's body and dispensing the photoreactive agent from the tip of the hypodermic needle into the target tissue.

4. The method of claim 1, wherein the photoreactive agent is locally delivered to the target tissue by disposing a photoreactive agent depot adjacent or within target tissue with emission of the photoreactive agent from the photoreactive agent depot into the target tissue.

5. The method of claim 4, wherein the photoreactive agent depot is comprised of a polymer impregnated with the photoreactive agent.

6. The method of claim 4, wherein the target tissue comprises an intracorporeal tumor and the photoreactive agent depot is disposed within the tumor.

7. The method of claim 1, wherein the photoreactive agent is locally delivered to the target tissue by a coronary delivery catheter and further comprising:
advancing a coronary catheter having an injection lumen and outlet ports into the patient's vasculature until the outlet ports are disposed adjacent the target tissue; and
injecting the photoreactive agent through the injection lumen and out of the outlet ports to the target tissue or tissue adjacent the target tissue.

8. The method of claim 7, wherein the coronary delivery catheter further comprises an expandable balloon on a distal end of the coronary delivery catheter with the outlet ports disposed on the expandable balloon and further comprising injecting the photoreactive agent through the injection lumen into the expandable balloon so as to expand the expandable balloon against the target tissue or tissue adjacent the target tissue and expel the photoreactive agent out of the outlet ports and into contact with the target tissue or tissue adjacent the target tissue.

9. The method of claim 1, wherein the photoreactive agent is locally delivered to the target tissue by a urinary delivery catheter and further comprising:
advancing the urinary delivery catheter having an injection lumen and outlet ports into the patient's urethra until the delivery ports are disposed adjacent the target tissue; and
injecting the photoreactive agent through the injection lumen and out of the outlet ports to the target tissue or tissue adjacent the target tissue.

10. The method of claim 9, wherein the urinary delivery catheter further comprises an expandable balloon on a distal end of the urinary delivery catheter and further comprising advancing the distal end of the urinary delivery catheter into the patient's bladder and expanding the expandable balloon in the patient's bladder prior to injecting the photoreactive agent through the injection lumen and out of the outlet ports and into contact with the target tissue or tissue adjacent the target tissue.

11. The method of claim 9, wherein the target tissue comprises the patient's prostate tissue and further comprising advancing the urinary delivery catheter into the patient's urethra until the outlet ports are adjacent the patient's prostate tissue prior to injecting the photoreactive agent into the injection lumen and out of the outlet ports.

12. The method of claim 1, wherein the photoreactive agent is locally delivered to the patient's retina.

13. The method of claim 4, wherein the photoreactive agent is locally delivered to the patient's retina by injection into the vitreous by a thin hypodermic needle.

14. The method of claim 13, wherein the needle has a diameter gauge of about 29 to about 31.

15. The method of claim 4, wherein the photoreactive agent is locally delivered to the patient's retina by positioning of a photoreactive agent depot adjacent the sclera of the patient's eye.

16. The method of claim 15, wherein the photoreactive agent depot is comprised of a polymer impregnated with the photoreactive agent.

17. The method of claim 12, wherein the photoreactive agent is locally delivered to the patient's retina by gas jet injection adjacent the sclera of the patient's eye.

18. The method of claim 12, wherein the photoreactive agent is locally delivered to the patient's retina by an application of a contact disk disposed on the cornea of the patient's eye.

19. The method of claim 18, wherein the contact disk comprises a polymer impregnated with the photoreactive agent.

20. The method of claim 19, wherein the contact disk further comprises a first electrical lead extending from the contact disk to a voltage source which is in electrical communication with the patient's eye and transfer of the photoreactive agent from the contact disk to the patient's retina is facilitated by the application of a voltage between the contact disk and the patient's eye by the voltage source.

21. The method of claim 12, wherein the photoreactive agent is locally delivered to the patient's retina by the application of the photoreactive agent to the patient's eye in conjunction with ultrasonic energy being delivered to the patient's eye adjacent the photoreactive agent.

22. The method of claim 1, wherein the photoreactive agent is selected from indocyanine green, toluidine blue, aminolevulinic acid, texaphyrins, benzoporphyrins, phenothiazines, phthalocyanines, porphyrins, chlorins, purpurins, purpurinimides, bacteriochlorins, pheophorbides, pyropheophorbides and cationic dyes.

23. The method of claim 1, wherein the photoreactive agent is mono-L-aspartyl chlorin e6.

24. The method of claim 1, wherein photoactivating the photoreactive agent of the target tissue with electromagnetic radiation comprises activating at least one light source.

25. The method of claim 24, wherein the at least one light source comprises one of a light-emitting diode, laser diode, incandescent light bulb, gas discharge device, polymeric electroluminescent device, halogen bulb, chemical luminescence, vacuum fluorescence, radio frequency excited gas, microwave excited gas, and cold cathode fluorescent tube.

26. A method of performing photodynamic therapy on an eye of a patient comprising:
a) administering a photoreactive agent to the patient's body;
b) allowing the photoreactive agent to absorb into at least a portion of the patient's retina;
c) illuminating the retina of the patient with a fluorescence generating light so that the photoreactive agent in the patient's retina fluoresces and emits fluorescent light;
d) detecting the fluorescent light emitted from the patient's retina with a fluorescence detector capable of spatially segregating the location of a point source of fluorescent light from different points in the patient's retina and storage of fluorescent response data from various points of the patient's retina;
e) processing the fluorescence response date and generating a map of at least a portion of the patient's retina so as to create a map of the fluorescence response of the patient's retina indicating at least one location of abnormality on the patient's retina; and
f) delivery of photoreactive light targeted to the at least one location of abnormality on the patient's retina.

27. The method of claim 26, wherein the at least one location of abnormality on the patient's retina is indicated by the detection of supra-threshold photoreactive agent concentrations in the tissue at the location of abnormality.

28. The method of claim 26, wherein the photoreactive agent is locally delivered to the patient's retina.

29. The method of claim 28, wherein the photoreactive agent is locally delivered to the patient's retina by injection into the vitreous by a thin hypodermic needle.

30. The method of claim 29, wherein the needle has a diameter gauge of about 29 to about 31.

31. The method of claim 28, wherein the photoreactive agent is locally delivered to the patient's retina by positioning of a photoreactive agent depot adjacent the sclera of the patient's eye.

32. The method of claim 31, wherein the photoreactive agent depot is comprised of a polymer impregnated with the photoreactive agent.

33. The method of claim 28, wherein the photoreactive agent is locally delivered to the patient's retina by gas jet injection adjacent the sclera of the patient's eye.

34. The method of claim 28, wherein the photoreactive agent is locally delivered to the patient's retina by an application of a contact disk disposed on the cornea of the patient's eye.

35. The method of claim 34, wherein the contact disk comprises a polymer impregnated with the photoreactive agent.

36. The method of claim 35, wherein the contact disk further comprises a first electrical lead extending from the contact disk to a voltage source which is in electrical communication with the patient's eye and transfer of the photoreactive agent from the contact disk to the patient's retina is facilitated by the application of a voltage between the contact disk and the patient's eye by the voltage source.

37. The method of claim 28, wherein the photoreactive agent is locally delivered to the patient's retina by the application of the photoreactive agent to the patient's eye in conjunction with ultrasonic energy being delivered to the patient's eye adjacent the photoreactive agent.

38. The method of claim 26, wherein the at least one location of abnormality comprises age-related macular degeneration.

39. The method of claim 26, wherein the at least one location of abnormality comprises diabetic retinopathy.

40. The method of claim 26, further comprising evaluation of a treatment response of the patient's retina using real-time monitoring of fluorescence signal intensity as an indicator of vascular leakage.

41. The method of claim 26, wherein the photoreactive agent is selected from indocyanine green, toluidine blue, aminolevulinic acid, texaphyrins, benzoporphyrins, phenothiazines, phthalocyanines, porphyrins, chlorins, purpurins, purpurinimides, bacteriochlorins, pheophorbides, pyropheophorbides and cationic dyes.

42. The method of claim 26, wherein the photoreactive agent is mono-L-aspartyl chlorin e6.

43. The method of claim 26, wherein delivery of photoreactive light is accomplished by activating at least one light source comprised of one of a light-emitting diode, laser diode, incandescent light bulb, gas discharge device, polymeric electroluminescent device, halogen bulb, chemical luminescence, vacuum fluorescence, radio frequency excited gas, microwave excited gas, and cold cathode fluorescent tube.

44. A system for performing photodynamic therapy on a patient's retina comprising:
a) a source of fluorescence generating light configured to illuminate the retina of the patient;
b) a fluorescence detector configured to detect fluorescent light emanating from the retina of the patient;
c) a source of photoactivating light configured to deliver photoactivating light to the patient's retina; and
d) a processor programmed to accumulate, store and analyze fluorescence response data from the fluorescence detector in response to fluorescent light from the patient's retina and generate a map of the patient's retina based on the fluorescence data indicating locations of tissue abnormality and thereafter direct light from the source of photoactivating light which is targeted to the locations of tissue abnormality in the patient's retina.

45. The system of claim 44, wherein the source of fluorescence generating light comprises a laser having a characteristic wavelength of about 600 to about 700 nanometers.

46. The system of claim 44, wherein the source of fluorescence generating light comprises a laser having a characteristic wavelength of about 660 to about 670 nanometers.

47. The system of claim 44, wherein the source of photoactivating light comprises a laser having a characteristic wavelength of about 500 to about 800 nanometers.

48. The system of claim 47, wherein the source of photoactivating light comprises a laser having a characteristic wavelength of about 600 to about 700 nanometers.

49. The system of claim 47, wherein the source of photoactivating light comprises one of a light-emitting diode, laser diode, incandescent light bulb, gas discharge device, polymeric electroluminescent device, halogen bulb, chemical luminescence, vacuum fluorescence, radio frequency excited gas, microwave excited gas, and cold cathode fluorescent tube.

50. The method of claim 1, wherein the target tissue is or results from restenosis, atheroma, benign prostatic hypertropy, age-related macular degeneration, diabetic retinopathy or a tumor.

51. A device for performing photodynamic therapy on the eye of a patient, comprising:
an elongate arm, wherein at least a portion of the arm follows a curvature that substantially conforms to the curvature of the eye;
a photoactivating light source that emits light along a light path, the light source positioned at a distal end of the elongate arm, wherein the elongate arm is sized to be positioned adjacent an outer surface of the eye such that a target portion of the eye is positioned in the light path.

52. A device as defined in claim 51, wherein the light source is one of a light-emitting diode, laser diode, incandescent light bulb, gas discharge device, polymeric electroluminescent device, halogen bulb, chemical luminescence, vacuum fluorescence, radio frequency excited gas, microwave excited gas, and cold cathode fluorescent tube.

53. A device as defined in claim 51, additionally comprising a lens positioned in the light path, wherein the lens focuses light from the light source.

54. A device as defined in claim 51, wherein the arm follows a curvature defined by a radius, and wherein the radius is approximately 12 mm.

55. A device as defined in claim 51, wherein the light source emits light having a characteristic wavelength of about 500 to about 800 nanometers.

56. A device for delivering a photoreactive agent to the eye of a patient, comprising:
a hypodermic needle, wherein at least a portion of the needle follows a curvature that substantially conforms to the curvature of the eye, wherein the photoreactive agent can be dispensed from a distal end of the needle;
a sheath that at least partially surrounds the needle, wherein the sheath follows a curvature that substantially conforms to the curvature of the eye.

57. A device as defined in claim 56, wherein the needle can be retracted such that the distal end of the needle is contained within the sheath, and wherein the needle can be advanced so that the distal end of the needle protrudes outwardly from the sheath.

58. A device as defined in claim 57, wherein the distal end of the needle can only be advanced outwardly a fixed distance from a distal edge of the sheath.

59. A device as defined in claim 56, additionally comprising a syringe attached to the needle, wherein the syringe can be actuated to dispense the photoreactive agent through the distal end of the needle.

60. A device as defined in claim 56, wherein a flexible coupling attaches the needle to the syringe so that the needle can be moved to various orientations relative to the syringe.

61. A device as defined in claim 56, wherein the needle follows a curvature defined by a radius, and wherein the radius is approximately 12 mm.
